(19) United States
(12) Patent Application Publication
(10) Pub. No.: US 2018/0132825 A1
(43) Pub. Date: May 17, 2018

(54) Ultrasonic Diagnosis/Treatment Device and Ultrasonic Diagnosis/Treatment Method

(71) Applicants: SonoCore, Inc., Tokyo (JP); Fukuoka University, Fukuoka-shi, Fukuoka (JP)

(72) Inventor: Katsuro TACHIBANA, Fukuoka (JP)

(21) Appl. No.: 15/564,404

(22) PCT Filed: Apr. 6, 2016
(86) PCT No.: PCT/JP2016/061258

§ 371 Date: Oct. 4, 2017

(30) Foreign Application Priority Data
Apr. 6, 2015 (JP) 2015-077778

(51) Int. Cl. A61B 8/00; A61N 7/00; A61B 8/06; A61B 8/08

(52) U.S. Cl. CPC A61B 8/4488; A61N 7/00; A61B 8/54; A61B 8/488; A61B 8/06

(57) ABSTRACT

An ultrasonic diagnosis/treatment device includes a placement surface formed of an inner surface of a sphere; a plurality of ultrasonic transducers placed on the placement surface so that a focal point of an ultrasonic wave to be emitted is located at a position that is near an outside of an outer surface of a subject and is spaced apart from the outer surface by a predetermined distance; and a control part configured to control transmission and reception of each of the ultrasonic transducers. The control part includes a diagnosis mode in which at least one ultrasonic transducer is oscillated toward a diagnosis region within a subject and a reflected wave from the diagnosis region is received by the at least one ultrasonic transducer to visualize the reflected wave, and a treatment mode in which the at least one ultrasonic transducer is oscillated toward an inside of the subject.

### CROSS REFERENCE TO RELATED APPLICATIONS

This application is a filing under 35 U.S.C. 371 of International Application No. PCT/JP2016/061258 filed Apr. 6, 2016, entitled “Ultrasonic Diagnosis/Treatment Device and Ultrasonic Diagnosis/Treatment Method” which claims priority to Japanese Patent Application No. 2015/077778 filed Apr. 6, 2015, entitled “Ultrasonic Diagnosis/Treatment Device and Ultrasonic Diagnosis/Treatment Method.”

### TECHNICAL FIELD

The present disclosure relates to an ultrasonic diagnosis/treatment device that performs diagnosis and treatment on a subject by using an ultrasonic wave, and an ultrasonic diagnosis/treatment method.

### BACKGROUND ART

In recent years, ultrasonic diagnosis for in vivo diagnosis of a patient (subject) by using an ultrasonic wave, and an ultrasonic treatment to perform treatment on an affected area by using an ultrasonic wave have been widely utilized.

Furthermore, as disclosed in PTL 1 described below, an ultrasonic diagnosis/treatment device that performs an ultrasonic diagnosis as well as an ultrasonic treatment using the same device is known. The ultrasonic diagnosis/treatment device disclosed in PTL 1 includes a group of ultrasonic transducers for treatment that are disposed on an inner surface of a main body part having a spherical shell shape, and an ultrasonic probe for diagnosis that is disposed at a central part of the main body part having a spherical shell shape. The group of ultrasonic transducers for treatment is disposed on the inner surface of the main body part having a spherical shell shape, thereby making it possible to form a focal point at one point in vivo. The ultrasonic probe for diagnosis enables in vivo diagnosis by mechanical scan or electronic scan.

### CITATION LIST

### Patent Literature

[PTL 1] Japanese Unexamined Patent Application, Publication No. 2001-70333

### SUMMARY OF INVENTION

### Technical Problem

However, in the ultrasonic diagnosis/treatment device disclosed in the Patent Literature described above, the group of ultrasonic transducers for treatment and the ultrasonic probe for diagnosis are separately provided. Accordingly, it is necessary to use different types of ultrasonic elements, which causes a problem that the device configuration is complicated and the cost thereof increases.

Furthermore, the ultrasonic probe for diagnosis disclosed in the Patent Literature described above is disposed at the central part of the main body part having a spherical shell shape. Accordingly, if the region through which an ultrasonic wave can be effectively transmitted is narrow, a wide field-of-view angle cannot be ensured in vivo and thus a satisfactory diagnosis cannot be performed. For example, in the case of diagnosing a brain, the ultrasonic wave, which greatly attenuates in a bone, is transmitted using a limited thin region of the bone of the skull. However, if the ultrasonic probe is located immediately above the region through which the ultrasonic wave can be transmitted, or immediately above a normal axis direction, like in the ultrasonic probe disclosed in the Patent Literature described above, it is difficult to ensure a sufficient field-of-view angle toward the inside of the brain from the region.

Furthermore, the group of ultrasonic transducers for treatment disclosed in the Patent Literature described above is disposed on the inner surface of the main body part having a spherical shell shape, thereby forming a focal point at one point in the body. However, as described above, when the ultrasonic wave from a limited region through which the ultrasonic wave is transmitted, the region on which the ultrasonic wave is irradiated on the surface of the body of a patient may be larger than the region through which the ultrasonic wave is transmitted. In such a case, ultrasonic waves emitted from ultrasonic transducers disposed on the outer peripheral side of the main body part having a spherical shell shape are blocked on the outside of the region through which the ultrasonic wave can be transmitted (e.g., blocked by a thick bone), which makes it difficult to effectively use these ultrasonic transducers.

The present disclosure has been made in view of the above-mentioned circumstances, and an object is to provide an ultrasonic diagnosis/treatment device and an ultrasonic diagnosis/treatment method which are capable of irradiating an ultrasonic wave from the outside of a subject to perform in vivo diagnosis and perform treatment on an affected area, even when the region on which the ultrasonic wave can be irradiated is limited.

### Solution to the Problem

The ultrasonic diagnosis/treatment device according to the present disclosure includes: a plurality of ultrasonic transducers placed in such a manner that a focal point of an ultrasonic wave to be emitted is located at a position that is near an outside of an outer surface of a subject and is spaced apart from the outer surface by a predetermined distance; and a control part configured to control transmission and reception of each of the ultrasonic transducers. The control part includes: a diagnosis mode in which at least one of the ultrasonic transducers is oscillated toward a diagnosis region within the subject and a reflected wave from the diagnosis region is received by the at least one of the ultrasonic transducers to visualize the reflected wave; and a treatment mode in which the at least one of the ultrasonic transducers is oscillated toward an inside of the subject.

The plurality of ultrasonic transducers is disposed in such a manner that the focal point is located at a position that is near an outside of an outer surface of a subject and is spaced apart from the outer surface by a predetermined distance. With this configuration, the ultrasonic wave emitted from each of the ultrasonic transducers can be prevented from being concentrated on one location on the outer surface of the subject or within the body of the subject, so that there is no thermal adverse effect, such as burn, on the subject.

Furthermore, the focal point is formed at a predetermined position near the outside of the outer surface of the subject, thereby reducing the ultrasonic wave irradiation region on the outer surface of the subject as much as possible, and making it possible to perform diagnosis or treatment by using all ultrasonic transducers effectively even when the region through which the ultrasonic wave can be irradiated on the subject is limited.

Note that the “focal point” according to the present disclosure is preferably as close to the outer surface of the subject as possible, and is located at a position spaced apart from the outer surface of the subject so as to prevent an adverse effect, such as burn, caused due to the concentration of the ultrasonic wave emitted from each ultrasonic transducer on the focal point.

Furthermore, in the ultrasonic diagnosis/treatment device according to the present disclosure, the plurality of ultrasonic transducers is placed on a placement surface which is a concave curve.

The plurality of ultrasonic transducers is placed on the placement surface, which is a concave curve, thereby making it possible to set the focal point at a position that is near the outside of the outer surface of the subject and is spaced apart from the outer surface by a predetermined distance.

The concave curve is not particularly limited as long as the focal point can be located at a desired position. For example, a partial rotor is used. The partial rotor indicates a shape that is formed by rotating a predetermined arc or line about a central axis, in order words, a part obtained by cutting a rotor along a plane perpendicular to the central axis. Specific examples of the partial rotor include a part of a sphere, and a part of a paraboloid of revolution.

Furthermore, in the ultrasonic diagnosis/treatment device according to the present disclosure, the predetermined distance is a range from 3 mm to 30 mm.

The predetermined distance is set in a range from 3 mm to 30 mm and the focal point position of the ultrasonic wave is set near the outer surface of the subject. Thus, an adverse effect, such as burn, on the subject can be prevented and the ultrasonic wave irradiation region can be reduced as much as possible.

The predetermined value is more preferably from 5 mm to 20 mm.

Furthermore, in the ultrasonic diagnosis/treatment device according to the present disclosure, the control part causes each of the ultrasonic transducers to be sequentially oscillated at a different time or at a different phase, in the diagnosis mode.

In the diagnosis mode, the ultrasonic transducers are sequentially oscillated at a different time or at a different phase. Thus, the reflected wave received by each ultrasonic transducer can be separated using an emission time or an emission phase, and thus can be easily visualized.

Note that when the ultrasonic transducers are caused to emit an ultrasonic wave at the same time, the oscillation frequency of each ultrasonic transducer is varied, to thereby make it possible to separate the reflected wave.

Furthermore, a control for oscillating all ultrasonic transducers at the same time and at the same oscillation frequency may be provided.

Furthermore, in the ultrasonic diagnosis/treatment device according to the present disclosure, a maximum spread angle of the ultrasonic wave at the focal point is in a range from 80° to 160°.

The maximum spread angle of the ultrasonic wave at the focal point is in a range from 80° to 160°. Thus, the ultrasonic wave can be irradiated with a sufficient spread angle toward the inside of the body of the subject, and a large diagnosis region and a large treatment region of the subject can be set. In particular, the use of an ultrasonic transducer with a large incident angle with respect to the outer surface of the subject (an angle formed between the incident ultrasonic wave and a normal axis direction on the outer surface) enables the ultrasonic wave to be reached even when the affected area is located on the outside of the irradiation area on the outer surface and at a shallow depth from the outer surface.

Note that the “spread angle” according to the present disclosure refers to an angle on both sides sandwiching a symmetric axis (central axis) passing through the focal point. The term “maximum spread angle” refers to a spread angle that can be formed when all ultrasonic transducers are used. The maximum spread angle is more preferably in a range from 100° to 140°.

Furthermore, in the ultrasonic diagnosis/treatment device according to the present disclosure, in the diagnosis mode, the control part selects at least one ultrasonic transducer that irradiates an ultrasonic wave in a direction opposite to a movement direction of a flow of blood flowing within a blood vessel, oscillates the selected ultrasonic transducer toward the blood vessel to obtain ultrasonic wave Doppler, and measures a rate of the flow of the blood.

The plurality of ultrasonic transducers is disposed in such a manner that the focal point is located at a position that is near the outer surface of the subject (e.g., disposed on the placement surface which is a concave curve), thereby making it possible to irradiate the ultrasonic wave in various direction within the body of the subject. Accordingly, there are ultrasonic transducers capable of irradiating the ultrasonic wave in a direction opposite to a movement direction of a flow of blood flowing within a blood vessel of a measurement target, i.e., in a direction substantially parallel to the orientation of the blood flow, even when the blood vessel faces in various direction. At least one ultrasonic transducer that irradiates the ultrasonic wave in a direction opposite to the movement direction of the blood flow is selected to obtain ultrasonic wave Doppler. Since the ultrasonic wave is irradiated in the direction opposite to the movement direction of the blood flow, a clear Doppler shift can be obtained and the rate of the blood flow can be measured with a high accuracy.

Furthermore, in the ultrasonic diagnosis/treatment device according to the present disclosure, the control part causes at least one of the ultrasonic transducers corresponding to a treatment position to be oscillated toward the treatment position where a drug and an ultrasonic treatment accelerating substance are administered in the treatment mode.

The drug and the ultrasonic treatment accelerating substance are administered at the treatment position, and at least one ultrasonic transducer corresponding to the treatment position is oscillated. As a result, the ultrasonic treatment accelerating substance using the non-thermal effect of the ultrasonic wave accelerates the penetration of the drug to the treatment position. In this manner, the present disclosure can be applied not only to a thermal treatment using heat due to a thermal energy of an ultrasonic wave, but also to a non-thermal treatment that accelerates the drug effect.

Examples of the ultrasonic treatment accelerating substance include microbubbles used as ultrasonic wave contrast media. Such microbubbles include a large number of microcapsules each containing a gas and having a diameter of about 0.1 μm to 100 μm.

Furthermore, the ultrasonic diagnosis/treatment device according to the present disclosure is characterized by including the deformable contact part that is in contact with the surface of the subject and is elastically deformable.

Since the deformable contact part that is in contact with the surface of the subject and is elastically deformable is provided, the orientation of each ultrasonic transducer can be changed as appropriate depending on the position of the affected area on which diagnosis or treatment is performed.

Furthermore, in the ultrasonic diagnosis/treatment device according to the present disclosure, the control part includes, in the treatment mode, a frequency sweep mode for sweeping frequencies included in pulse waves oscillated from the ultrasonic transducers from a high frequency region to a low frequency region.

The frequency sweep mode in which the frequency included in the pulse wave oscillated from each ultrasonic transducer is swept from a high frequency region to a low frequency region enables a large number of cells to be killed at the treatment position.

Note that the above-mentioned frequency sweep mode can also be applied to a publicly-known ultrasonic wave and treatment apparatus. Specifically, the present disclosure is not limited to the ultrasonic treatment device including a plurality of ultrasonic transducers placed in such a manner that the focal point of the ultrasonic wave to be emitted is located at a position that is near the outside of the outer surface of the subject and is spaced apart from the outer surface by a predetermined distance, but also can be applied to an ultrasonic treatment device simply including an ultrasonic transducer.

Furthermore, the ultrasonic diagnosis/treatment device according to the present disclosure is characterized by including the ultrasonic transducer placement shape change part that changes the shape of the placement of each of the ultrasonic transducers so as to change the maximum spread angle of the ultrasonic wave at the focal point.

The maximum spread angle of the ultrasonic wave at the focal point can be changed by the shape of changing the placement shape of the ultrasonic transducers. Thus, a desired field-of-view range or treatment range can be ensured depending on an area on which diagnosis or treatment is performed.

Furthermore, the ultrasonic diagnosis/treatment method according to the present disclosure uses the ultrasonic diagnosis/treatment device that includes a plurality of ultrasonic transducers placed in such a manner that a focal point of an ultrasonic wave to be emitted is located at a position that is near the outside of the outer surface of the subject and is spaced apart from the outer surface by a predetermined distance, the ultrasonic diagnosis/treatment method including: performing the diagnosis mode in which at least one of the ultrasonic transducers is oscillated toward a diagnosis region within the subject and a reflected wave from the diagnosis region is received by the at least one of the ultrasonic transducers to visualize the reflected wave, and the treatment mode in which the at least one of the ultrasonic transducers is oscillated toward an inside of the subject.

The plurality of ultrasonic transducers is disposed in such a manner that the focal point is located at a position that is near the outside of the outer surface of the subject and is spaced apart from the outer surface by the predetermined distance. With this configuration, the ultrasonic wave emitted from each of the ultrasonic transducers can be prevented from being concentrated on one location on the outer surface of the subject or within the body of the subject, so that there is no thermal adverse effect, such as burn, on the subject.

Furthermore, the focal point is formed at a predetermined position near the outside of the outer surface of the subject, thereby reducing the ultrasonic wave irradiation region on the outer surface of the subject as much as possible, and making it possible to perform diagnosis or treatment by using all ultrasonic transducers effectively even when the region through which the ultrasonic wave can be irradiated on the subject is limited.

### Advantageous Effects of Invention

The ultrasonic transducers are placed in such a manner that the focal point of the ultrasonic wave to be emitted is located at a position that is near the outside of the outer surface of the subject and is spaced apart from the outer surface by a predetermined distance. Consequently, the ultrasonic wave can be irradiated from the outside of the subject to perform in vivo diagnosis and the affected area can be treated, even when the region through which the ultrasonic wave can be transmitted is limited.

### BRIEF DESCRIPTION OF DRAWINGS

FIG. 1 is a perspective view illustrating an ultrasonic diagnosis/treatment device according to an embodiment of the present disclosure.

FIG. 2 is a longitudinal sectional view illustrating the ultrasonic diagnosis/treatment device illustrated in FIG. 1.

FIG. 3 is a diagram illustrating a placement state of ultrasonic transducers.

FIG. 4 is a longitudinal sectional view illustrating a focal point connected by ultrasonic waves emitted from the ultrasonic transducers.

FIG. 5 is a perspective view illustrating a usage state of the ultrasonic diagnosis/treatment device illustrated in FIG. 1.

FIG. 6 is a view illustrating a state where an ultrasonic wave is irradiated through a thin part of a skull.

FIG. 7 is a graph illustrating an apoptosis when an ultrasonic wave is irradiated in a frequency sweep mode.

FIG. 8 is a graph illustrating a survival rate obtained during the experiment illustrated in FIG. 7.

FIG. 9 is a graph illustrating a survival rate when a pulse repetition frequency is changed.

FIG. 10 is a graph illustrating a survival rate when an ultrasonic wave output is changed when the pulse repetition frequency is 0.5 Hz and the irradiation time is 180 seconds.

FIG. 11 is a graph illustrating a survival rate when the ultrasonic wave output is changed when the pulse repetition frequency is 50 Hz and the irradiation time is 180 seconds.

FIG. 12 is a graph illustrating a survival rate when the ultrasonic wave output is changed when the pulse repetition frequency is 0.5 Hz and the irradiation time is 90 seconds.

FIG. 13 is a graph illustrating a survival rate when the ultrasonic wave output is changed when the pulse repetition frequency is 50 Hz and the irradiation time is 90 seconds.

FIG. 14 is a graph illustrating a survival rate and an apoptosis when an ultrasonic wave is irradiated in the frequency sweep mode.

FIG. 15 is a graph illustrating a survival rate when microbubbles are used, and a survival rate when microbubbles are not used.

FIG. 16 is a graph illustrating a rate of cell killing when Sonazoid MB is used and the center frequency 455 kHz.

FIG. 17 is a graph illustrating a rate of cell killing when Sonazoid MB is used and the center frequency is 1.5 MHz.

FIG. 18 is a graph illustrating a rate of cell killing when Sonazoid MB is not used and the center frequency is 455 kHz.

FIG. 19 is a graph illustrating a rate of cell killing when Sonazoid MB is not used and the center frequency is 1.5 MHz.

FIG. 20 is a graph illustrating a rate of cell killing when Sonazoid MB is used, the center frequency is 1.5 MHz, and PRF is 10 Hz.

FIG. 21 is a graph illustrating a rate of cell killing when Sonazoid MB is used, the center frequency is 1.5 MHz, and PRF is 50 Hz.

FIG. 22 is a graph illustrating a rate of cell killing when Sonazoid MB is used, the center frequency is 1.5 MHz, and PRF is 100 Hz.

FIG. 23 is a graph illustrating a rate of cell killing when Sonazoid MB is used and the input voltage is 15 V at a resonance frequency.

FIG. 24 is a graph illustrating a rate of cell killing when Sonazoid MB is used and the input voltage is 20 V at a resonance frequency.

FIG. 25 is a graph illustrating an average value of FITC fluorescence intensities.

FIG. 26 is a graph illustrating a rate of cell killing depending on whether or not a frequency sweep is present.

FIG. 27 is a plan view illustrating a pallet used for experiments.

FIG. 28 is a longitudinal sectional view illustrating an enlarged view of a single well.

FIG. 29 is a plan view illustrating an experimental jig.

FIG. 30 is a partial longitudinal sectional view illustrating a section taken along a line A-A in FIG. 29.

FIG. 31 is a diagram illustrating a modified example of sequentially oscillating the ultrasonic transducers.

FIG. 32 is a diagram illustrating a modified example of sequentially oscillating the ultrasonic transducers.

FIG. 33 is a longitudinal sectional view illustrating a modified example of a method of installing the ultrasonic transducers.

FIG. 34 is a longitudinal sectional view illustrating a modified example using an elastically deformable coupling.

FIG. 35 is a perspective view illustrating a modified example in which ultrasonic transducers are placed in a cylindrical surface shape.

FIG. 36 is a perspective view illustrating a modified example in which ultrasonic transducers are placed in an elliptic surface shape.

FIGS. 37(a) and 37(b) illustrate a state in which the curvature radius of an inner surface on which ultrasonic transducers are placed is changed; FIG. 37(a) is a longitudinal sectional view illustrating a state in which the curvature radius is relatively large, and FIG. 37(b) is a longitudinal sectional view illustrating a state in which the curvature radius is relatively small.

### DESCRIPTION OF EMBODIMENTS

Embodiments of the present disclosure will be described below with reference to the drawings.

FIG. 1 illustrates an ultrasonic diagnosis/treatment device 1 according to this embodiment.

The ultrasonic diagnosis/treatment device 1 includes an ultrasonic diagnosis/treatment probe 3 (hereinafter referred to as the “probe 3”) and a control part 5 that performs, for example, control of transmission and reception of ultrasonic transducers.

The probe 3 includes an ultrasonic wave transmission/reception part 7 in which a plurality of ultrasonic transducers is disposed, and a coupling part 9 serving as an acoustic matching layer.

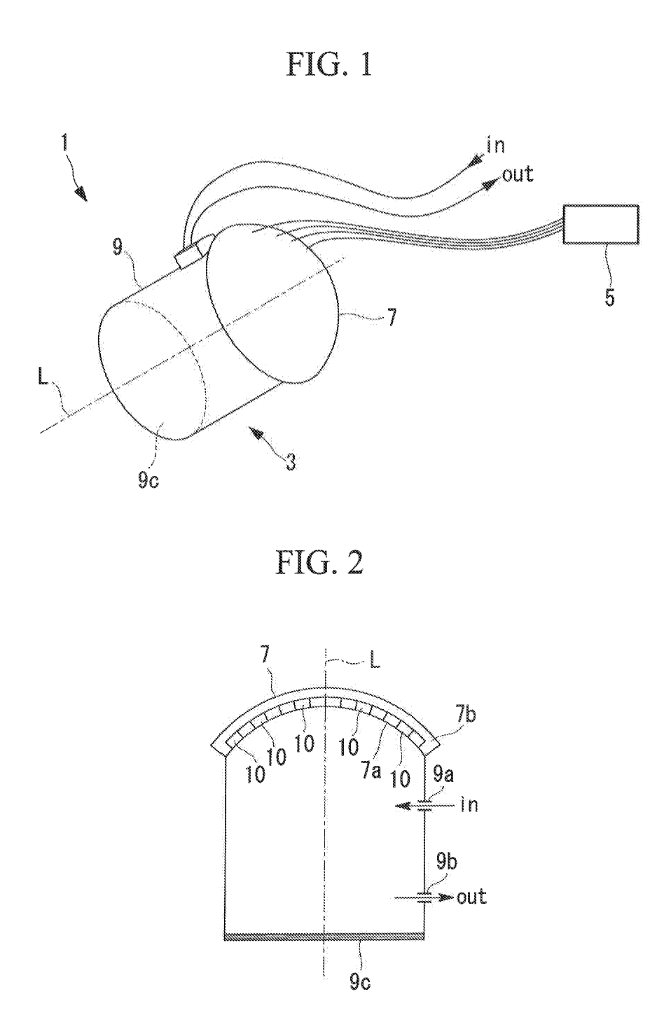

As illustrated in FIG. 2, the ultrasonic wave transmission/reception part 7 has a dome shape including an inner surface 7a which is a spherical concave curve. Note that the inner surface 7a is not limited to the spherical shape, but instead the inner surface may have various curved surfaces as long as the inner surface forms a concave curve. For example, the inner surface may be a curved surface defined by a part of a rotor formed about the central axis (specifically, the central axis of the coupling part 9 having a cylindrical shape) L of the probe 3 as a rotation axis. Accordingly, examples of the inner surface include other curved surfaces such as a paraboloid, a cylindrical surface, and an elliptic surface.

A plurality of ultrasonic transducers 10 is disposed on the inner surface 7a. Specifically, the inner surface 7a is a placement surface on which the plurality of ultrasonic transducers 10 is placed. The ultrasonic transducers 10 are piezoelectric elements. Typically, PZT (lead zirconate titanate) is used. Each ultrasonic transducer 10 is connected to the control part 5 and is caused to operate as a transmitter and a receiver by the control part 5. Specifically, each ultrasonic transducer operates as a transmitter in the treatment mode, and operates as a transmitter and a receiver in the diagnosis mode.

Note that as the ultrasonic transducers 10, cMUTs (Capacitive Micro-machined Ultrasonic Transducers) may be used instead of PZT. The cMUTs, which are capacitive ultrasonic transducers based on semiconductor technology, are capable of transmitting receiving ultrasonic wave frequencies in a wide frequency range, and have excellent acoustic characteristics. The cMUTs are created by patterning a large number of small sensors (cMUT cells) on a silicon substrate by a lithography technique.

A packing part 7b is provided on the back surface (upper surface in FIG. 2) of each ultrasonic transducer 10. The packing part 7b suppresses an extra vibration of the ultrasonic transducers 10, and transmits the ultrasonic vibration to the subject (coupling part 9) efficiently.

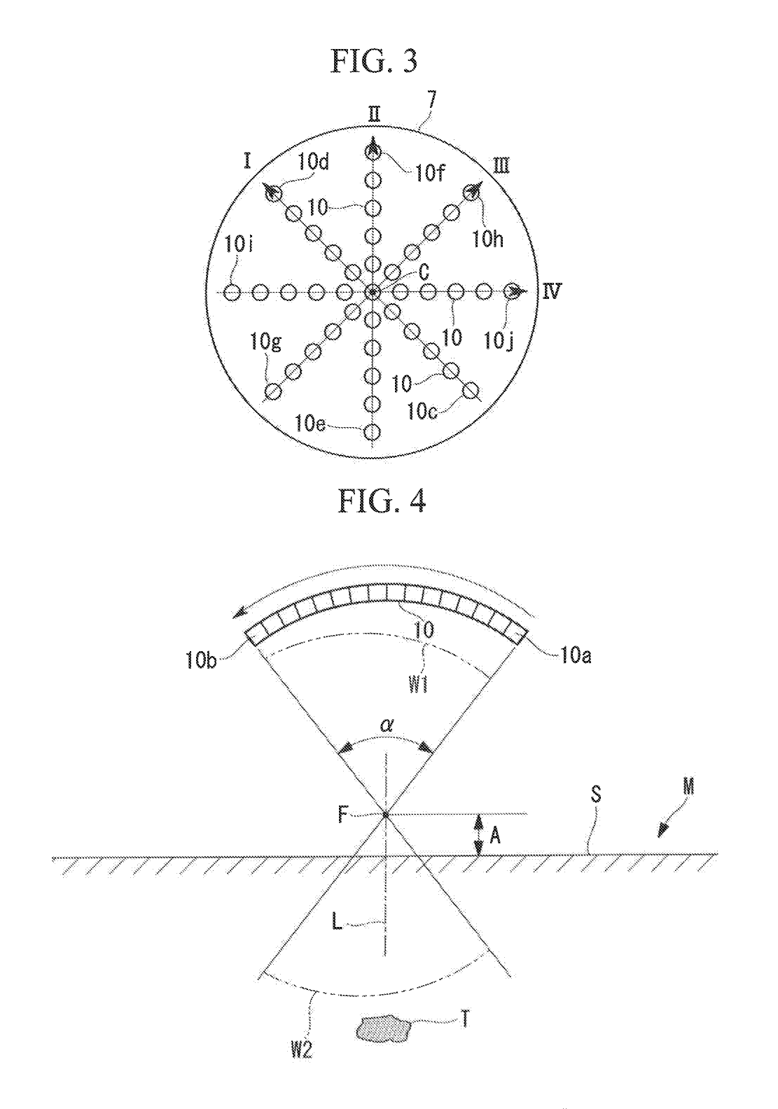

As illustrated in FIG. 3, the plurality of ultrasonic transducers 10 is radially arranged from a center C of the ultrasonic wave transmission/reception part 7 as a starting point when the ultrasonic wave transmission/reception part 7 as viewed from the bottom surface (i.e., as viewed from the concave side of the ultrasonic wave transmission/reception part 7). Note that in FIG. 3, the ultrasonic transducers 10 are placed in eight directions at every 45° from the center C. The directions in which the ultrasonic transducers are placed are not particularly limited. The ultrasonic transducers may be placed in at least four directions (i.e., crosswise), or may be placed in nine or more directions. Furthermore, the ultrasonic transducers may be concentrically placed about the center C.

FIG. 4 illustrates a position of a focal point F that is formed by the ultrasonic transducers 10 placed as described above. Note that as illustrated in FIG. 4, for ease of understanding, the coupling part 9 illustrated in FIGS. 1 and 2 is omitted. As illustrated in FIG. 4, the focal point F is set near the outside of an outer surface S of a subject M and is spaced apart from the outer surface S by a predetermined distance A. The predetermined distance A is preferably a distance close to the outer surface S so as not to be located on the outer surface S, and is, for example, from 3 mm to 30 mm, and preferably from 5 mm to 20 mm. Note that reference symbol T illustrated in FIG. 4 is a treatment target (treatment position) such as a tumor.

A maximum spread angle α of the ultrasonic wave at the focal point is from 80° to 160°, and preferably from 100° to 140°. The maximum spread angle α indicates an angle formed between both sides sandwiching a symmetric axis (central axis L) passing through the focal point F, and indicates a spread angle that can be formed when all ultrasonic transducers are used.

As illustrated in FIGS. 1 and 2, the coupling part 9 has a substantially cylindrical shape, and matches an acoustic impedance between the subject M and the ultrasonic transducers 10. The inside of the coupling part 9 is filled with an acoustic matching liquid such as deaerated water or silicone oil. As illustrated in FIG. 2, at a side part of the coupling part 9, an in-flow port 9a through which the acoustic matching liquid, such as deaerated water or silicone oil, flows into the coupling part 9, and an out-flow port 9b through which the acoustic matching liquid flows out from the coupling part 9, are provided. Through the in-flow port 9a and the out-flow port 9b, the acoustic matching liquid, such as deaerated water or silicone oil, is circulated from an external supply source (not illustrated). Note that the coupling part 9 is not limited to the acoustic matching liquid, such as deaerated water or silicone oil, like in this embodiment, as long as the coupling part is formed of a material that matches the acoustic impedance between the subject M and the ultrasonic transducers 10. Other materials other than liquid, such as a gel or solid may be used, as long as the acoustic matching layer is formed.

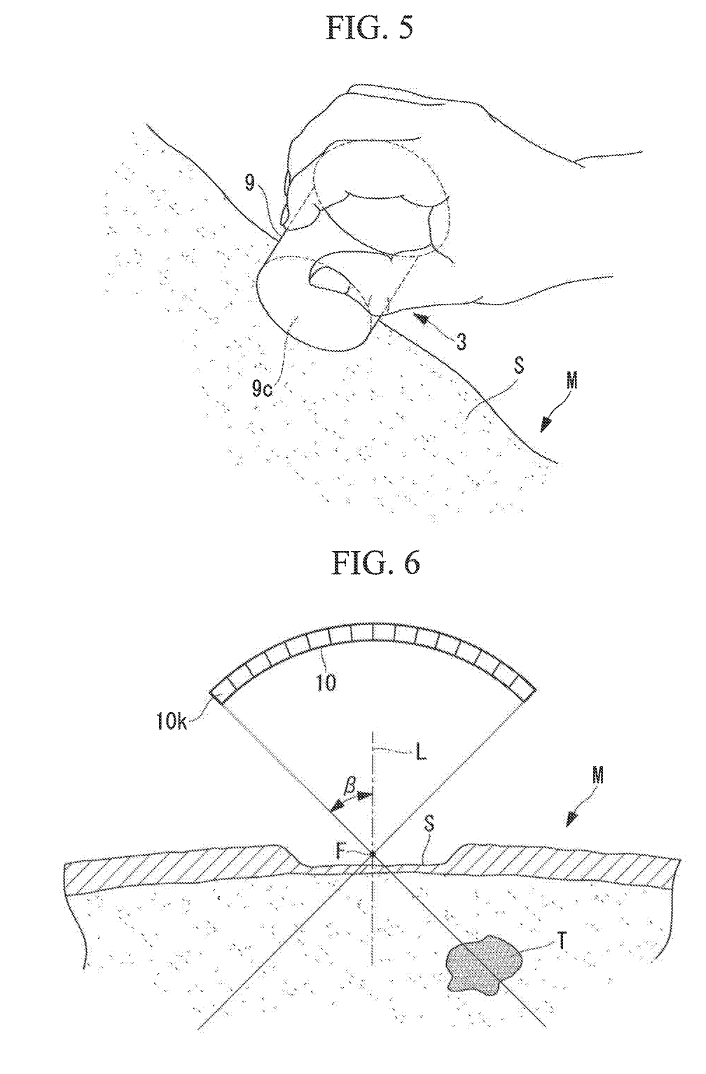

As illustrated in FIG. 5, the ultrasonic diagnosis/treatment device 1 can be carried around by gripping the probe 3 with a hand, and a bottom surface 9c of the coupling part 9 is installed at a location where diagnosis or treatment is performed. Thus, the bottom surface 9c of the coupling part 9 is a contact surface that is in direct contact with the outer surface S of the subject M. The ultrasonic wave from the ultrasonic transducers 10 for treatment or diagnosis is guided into the body of the subject M through the bottom surface 9c, and the reflected wave reflected from the inside of the body of the subject M is guided to the ultrasonic transducers 10 each serving as a receiver. Accordingly, the predetermined distance A illustrated in FIG. 4 is a distance from the bottom surface 9c of the coupling part 9 to the focal point F.

As illustrated in FIG. 1, the control part 5 is connected to the ultrasonic wave transmission/reception part 7, and controls each of the ultrasonic transducers 10. Specifically, the ultrasonic transducers 10 are oscillated based on an instruction from the control part 5, and a reflected signal from the subject M received by each ultrasonic transducer 10 is transmitted to the control part 5.

The control part 5 can adjust the frequency, output, and the like of each ultrasonic transducer 10, and can also adjust a pattern of a sequence of oscillation by each ultrasonic transducer 10. The frequency and output of each ultrasonic transducer 10 may be adjusted individually for each ultrasonic transducer 10. Furthermore, the frequency and output of each ultrasonic transducer 10 may be changed by the diagnosis mode and the treatment mode. For example, the output may be reduced in the diagnosis mode, and the output may be increased in the treatment mode.

Furthermore, the control part 5 performs processing for performing a predetermined calculation based on the reflected wave received by each ultrasonic transducer 10 and visualizing the reflected wave. As visualized images, various images, such as A mode image, B mode image, M mode image, and color Doppler, can be obtained. As an image processing technique, Ultrafast Imaging proposed by Mickael Tanter may be used (e.g., ‘Ultrafast Imaging in Biomedical Ultrasound’, IEEE Transactions on Ultrasonics, Ferroelectrics and Frequency Control, vol. 61, no. 1, pp. 102-119 January 2014).

The control part 5 is connected to a monitor (not illustrated) as an image display device, and various images as mentioned above are displayed.

The control part 5 includes, for example, a CPU (Central Processing Unit), a RAM (Random Access Memory), a ROM (Read Only Memory), and a computer readable storage medium. A series of processes for implementing various functions are stored in a storage medium or the like in the format of, for example, a program. This program is read out into a RAM or the like by a CPU, and information processing/operation processing is executed to thereby implement various functions. As the program, a form that is installed in advance in a ROM or other storage media, a form that is provided in a state of being stored in a computer readable storage medium, a form that is distributed through a communication means with a wire or wirelessly, or the like may be applied. Examples of the computer readable storage medium include a magnetic disk, magneto-optical disk, a CD-ROM, a DVD-ROM, and a semiconductor memory.

Next, an operation of the ultrasonic diagnosis/treatment device 1 described above will be described.

### Diagnosis Mode

In the diagnosis mode, the ultrasonic transducers 10 are sequentially oscillated with a predetermined time difference or phase difference according to an instruction from the control part 5. For example, as illustrated in FIG. 4, the ultrasonic transducers are sequentially oscillated in order from an ultrasonic transducer 10a, which is located at one end of the ultrasonic transducers, to an ultrasonic transducer 10b, which is located at the other end of the ultrasonic transducers. In this manner, the ultrasonic transducers 10 are oscillated in the diameter direction of the ultrasonic wave transmission/reception part 7, and the oscillation is sequentially repeated in different diameter directions. Specifically, as illustrated in FIG. 3, first, the ultrasonic transducers are sequentially oscillated in order from an ultrasonic transducer 10c, which is located at a lower right position in the figure, to an ultrasonic transducer 10d which is located at an upper left position (see reference symbol I in the figure). Next, the ultrasonic transducers are sequentially oscillated in order from an ultrasonic transducer 10e, which is located immediately below in the figure to an ultrasonic transducer 10f which is located immediately above (see reference symbol II in the figure). Next, the ultrasonic transducers are sequentially oscillated in order from an ultrasonic transducer 10g, which is located at an lower left position in the figure to an ultrasonic transducer 10h which is located at an upper right position (see reference symbol III in the figure). Lastly, the ultrasonic transducers are sequentially oscillated in order from an ultrasonic transducer 10i, which is located at the left end in the figure to an ultrasonic transducer 10j which is located at the right end (see reference symbol IV in the figure).

As described above, the ultrasonic wave is sequentially emitted from each ultrasonic transducer 10 with a predetermined time difference or phase difference. FIG. 4 illustrates an incident wavefront W1 of the ultrasonic wave emitted with the predetermined time difference or phase difference. The ultrasonic wave emitted from each ultrasonic transducer 10 passes through the focal point F, and then passes through the outer surface S of the subject M and enters into the body. Furthermore, the reflected wave reflected at each location in vivo is emitted from the outer surface S and received by each ultrasonic transducer 10. FIG. 4 illustrates a reflected wavefront W2 after the reflection in vivo. A reflected image obtained and received by each ultrasonic transducer 10 is an image that is symmetric to the focal point F. This reflected image is converted, as needed, by the control part 5, and the described diagnosis image as mentioned above is obtained.

### Blood Flow Test

The diagnosis mode includes a blood flow test for obtaining a blood flow rate within a blood vessel by grasping the reflected wave of the ultrasonic wave by erythrocytes.

The control part 5 grasps the position and orientation of the blood vessel in the diagnosis mode described above, and then one or more ultrasonic transducers 10 that emits an ultrasonic wave in a direction opposite to the orientation of the blood vessel to be diagnosis, i.e., in a direction substantially parallel to the orientation of the blood flow, are selected. Furthermore, the selected ultrasonic transducers 10 are oscillated to grasp the reflected wave reflected from the erythrocytes in the ultrasonic transducers 10 to measure the ultrasonic wave Doppler, thereby obtaining the blood flow rate. In this manner, the ultrasonic wave is irradiated in a direction in which Doppler shift by the ultrasonic wave is likely to be obtained, thereby obtaining the blood flow rate with a high accuracy.

### Treatment Mode

In the treatment mode, a predetermined ultrasonic transducer 10 is selected according to an instruction from the control part 5, and the ultrasonic wave is emitted from the selected ultrasonic transducer 10. Thus, as illustrated in FIG. 6, the ultrasonic wave is irradiated on the treatment target T, and treatment is performed. The selection of the ultrasonic transducer 10 for performing treatment may be performed in such a manner that the position of the treatment target T is grasped in the diagnosis mode mentioned above, and the ultrasonic transducers 10 disposed at the position corresponding to the treatment target T is selected. Alternatively, when the position of the treatment target T is recognized in advance, the diagnosis mode may be omitted.

In particular, as illustrated in FIG. 6, the use of an ultrasonic transducer 10k with a large incident angle (an angle β formed between the incident ultrasonic wave and the normal axis direction (central axis L in FIG. 6) on the outer surface) with respect to the outer surface S of the subject M enables the ultrasonic wave to reach the treatment target T that is located on the outside of the ultrasonic wave irradiation region on the outer surface S and at a shallow depth from the outer surface S.

Also in the diagnosis mode, the use of the ultrasonic transducer 10 with the large incident angle β enables diagnosis of the treatment target T that is located on the outside of the ultrasonic wave irradiation region on the outer surface S and at a shallow depth from the outer surface S.

### Non-Thermal Treatment

The present disclosure can be applied not only to a thermal treatment using heat due to a thermal energy of an ultrasonic wave, but also to a non-thermal treatment that accelerates a drug effect.

In the case of a non-thermal treatment, a drug and an ultrasonic treatment accelerating substance are administered to the treatment target T, and at least one ultrasonic transducer 10 (ultrasonic transducer 10k in FIG. 6) corresponding to the treatment target T is oscillated. As a result, the ultrasonic treatment accelerating substance using the non-thermal effect due to the ultrasonic wave energy accelerates the penetration of the drug to the treatment target T.

Examples of the ultrasonic treatment accelerating substance include microbubbles used as ultrasonic wave contrast media. Such microbubbles include a large number of microcapsules each containing a gas and having a diameter of about 0.1 μm to 100 μm.

### Frequency Sweep Mode

In the treatment mode, the following frequency sweep mode can be used.

In the frequency sweep mode, the frequency included in the pulse wave oscillated from the ultrasonic transducers 10 is swept from a high frequency region to a low frequency region. As a result, a larger number of cells can be killed at the treatment position than in a case where the frequency is swept from the low frequency region to the high frequency region. In the frequency sweep mode, for example, the sweep width is ±110 kHz at the center frequency of 510 kHz, and the frequency is swept in such a manner that the frequency decreases from 620 kHz to 400 kHz. The pulse repetition frequency is, for example, from 5 Hz to 50 Hz, and preferably in the vicinity of 10 Hz. The ultrasonic wave output is, for example, 30 mW/cm² or more, and preferably 80 mW/cm² or more. The irradiation time is, for example, 90 seconds or longer, and preferably 180 seconds or longer.

### EXPERIMENTAL RESULTS

Next, experimental results using the frequency sweep mode will be described.

The following experimental method was used.

<Cell Culture>

A human leukemia cell line U937 was used.

<Ultrasonic Wave Irradiation Method>

A 24-well culture plate (Lumox .A N.) was installed on the acoustic emitting surface of an ultrasonic transducer having an oscillator diameter of 20 mm through an acoustic coupling gel.

Each well is filled with 2 mL of 1 x106 cells/mL of cell suspension of a human leukemia cell line U937 prepared immediately before the irradiation of an ultrasonic wave. The culture plate was driven by an oscillator (SonoPore KTAC-4000, Nepagene) under a sine wave condition that the sweep width is ±110 kHz at the center frequency 510 kHz, the sweep interval is 0.2 ms, the pulse repetition frequency is 10 Hz, the duty ratio is 50%, and in a range from 20 mW/cm² to 80 mW/cm² (specifically, driven by the same oscillator in a range from 30 V to 60 V), and the ultrasonic wave is irradiated on U937 for 90 seconds at the ultrasonic wave intensity of 80 mW/cm². Effects of two different ultrasonic wave irradiation conditions, i.e., a case where the drive frequency is increased from 400 kHz to 620 kHz due to the frequency sweep (hereinafter referred to as “Sweep 1”), and a case where the drive frequency is decreased from 620 kHz to 400 kHz (hereinafter referred to as “Sweep 2”), on a rate of cell killing (comparison between survival rates before and after) and an apoptosis have been studied. Note that Sweep 2 corresponds to the frequency sweep mode of the present disclosure.

### Measurement of Cell Survival Rate

A trypan blue exclusion assay was used for life-and-death determination of U937 immediately after the ultrasonic wave exposure (n=4). In this case, n represents the number of experiments. After mixing and staining the same amount of Trypan Blue stain solution as the cell suspension, the number of living cells was measured by an automatic cell counter TC20 (Bio Rad). The cell survival rate was calculated from the ratio of the number of living cells obtained after the ultrasonic wave exposure to the number of living cells that are controlled without irradiation of the ultrasonic wave.

### Detection of Apoptosis (Programmed Cell Death)

The apoptosis of U937 on which the ultrasonic wave was irradiated was evaluated. After six hours from the ultrasonic wave exposure, the cells were double labeled with AnnexinV-Alexa and PI, and an early apoptosis and a late apoptosis were detected using an image-based cytometer (Tali, Life technologies).

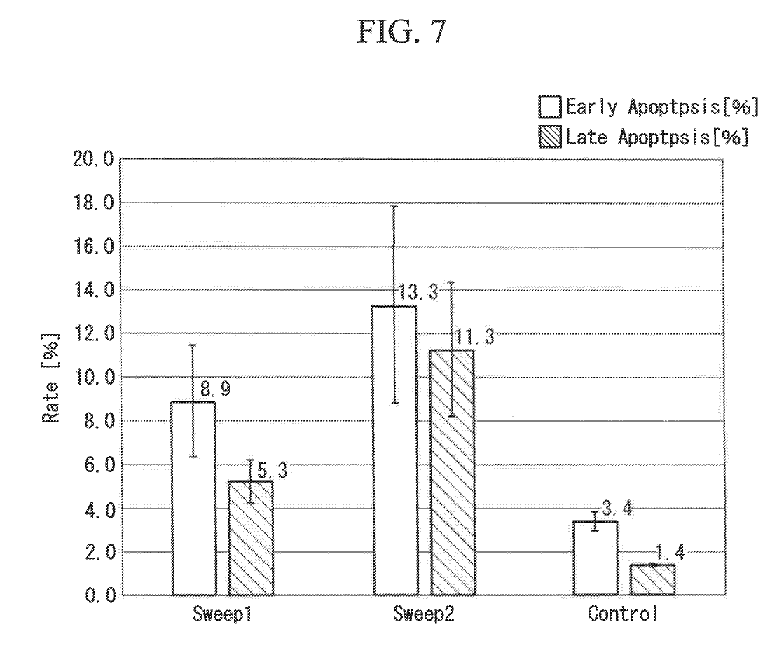

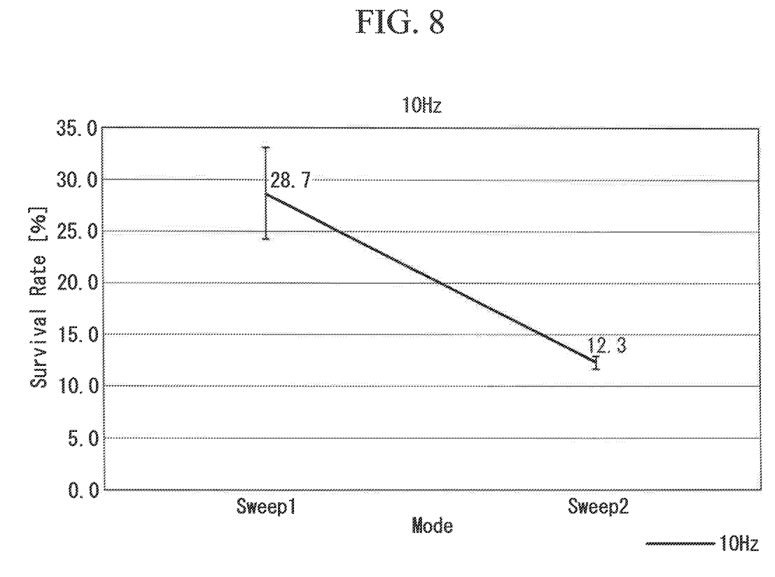

FIGS. 7 and 8 illustrate the experimental results under the conditions described above.

As illustrated in FIG. 7, the ultrasonic wave irradiation intensity in Sweep 1 is the same as the ultrasonic wave irradiation intensity in Sweep 2, but it was confirmed that the extent of apoptosis in Sweep 2 (the present disclosure) was more than the extent of apoptosis in Sweep 1.

Furthermore, as seen from FIG. 8, the survival rate of cancer cells in Sweep 2 (the present disclosure) was significantly lower than that in Sweep 1.

Furthermore, the results of measuring the survival rate by changing experimental conditions will be described.

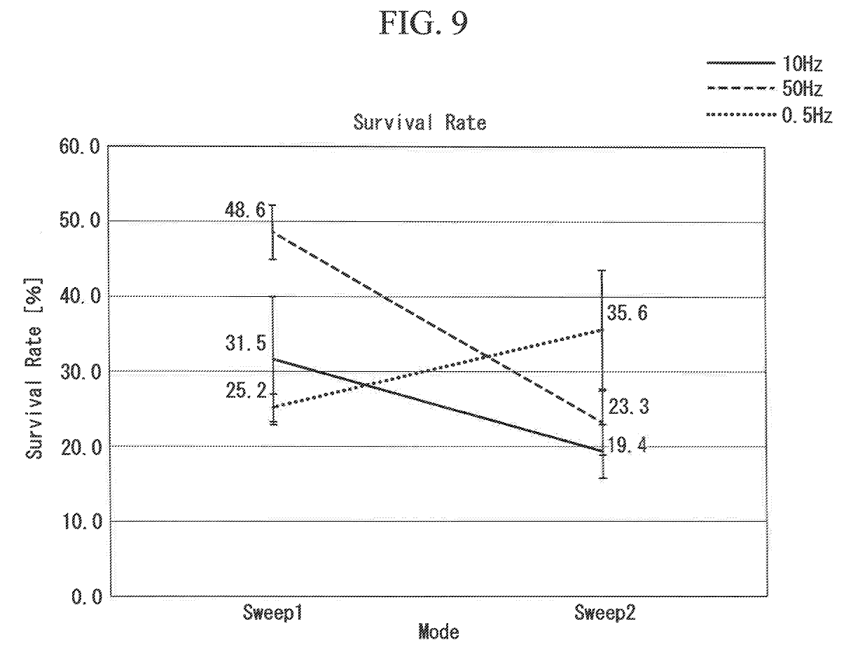

FIG. 9 illustrates the survival rate when the ultrasonic wave output is 80 mW/cm² and the pulse repetition frequency is changed to 0.5 Hz, 10 Hz, and 50 Hz under the irradiation condition of 180 seconds. As seen from FIG. 9, when the pulse repetition frequency is 10 Hz and 50 Hz, the survival rate in Sweep 2 (the present disclosure) is lower than that in Sweep 1. On the other hand, when the pulse repetition frequency is 0.5 Hz, the survival rate in Sweep 2 (the present disclosure) is higher than that in Sweep 1. Accordingly, when Sweep 2 is employed, the pulse repetition frequency is higher than 0.5 Hz, preferably equal to or higher than 5 Hz, and more preferably in the vicinity of 10 Hz since the survival rate at 10 Hz is lower than that at 50 Hz.

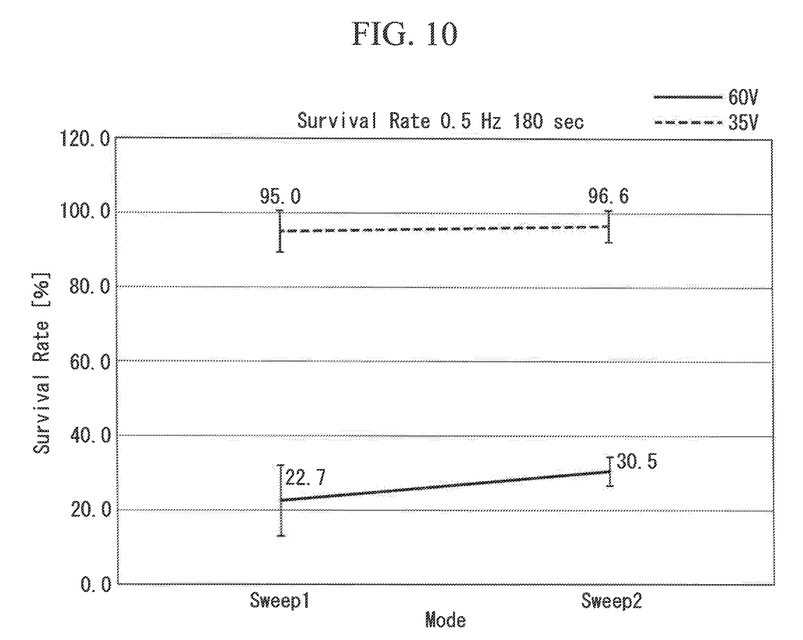

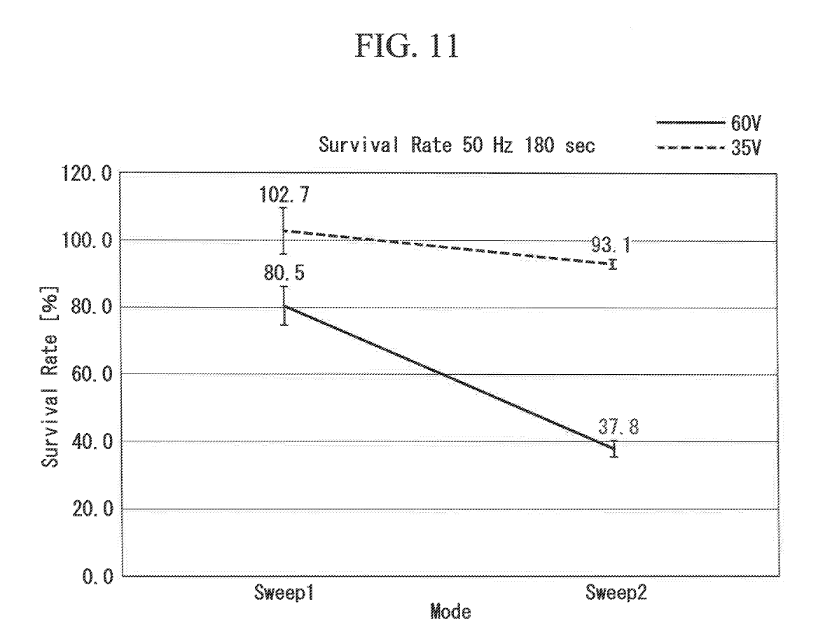

FIGS. 10 and 11 illustrate the survival rate when the ultrasonic wave output is changed to 35 V and 60 V when the irradiation time is set to 180 seconds. FIG. 10 illustrates the survival rate when the pulse repetition frequency is 0.5 Hz. FIG. 11 illustrates the survival rate when the pulse repetition frequency is 50 Hz. As seen from FIGS. 10 and 11, the survival rate at the ultrasonic wave output of 60 V is lower than that at 35 V, and when the ultrasonic wave output is 35 V, there is no significant difference between Sweep 1 and Sweep 2 (the present disclosure).

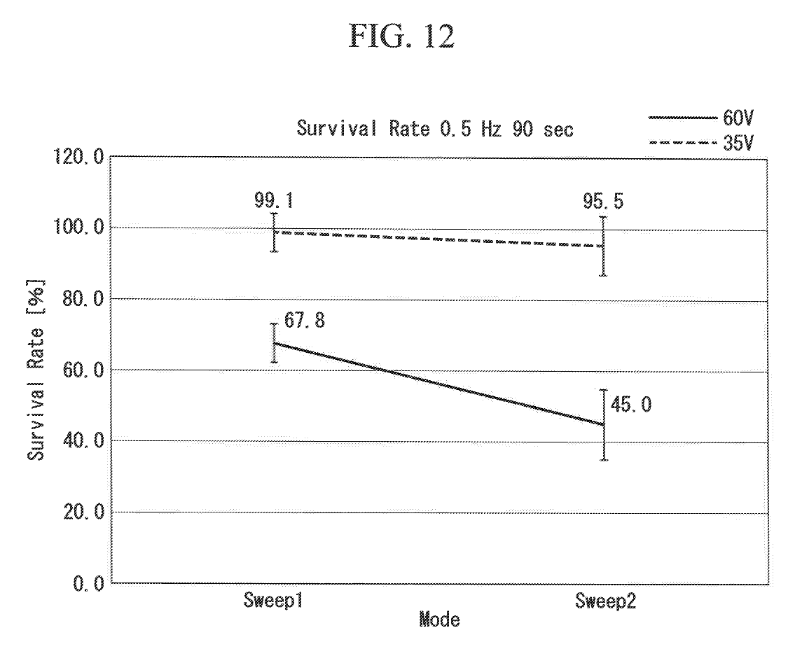

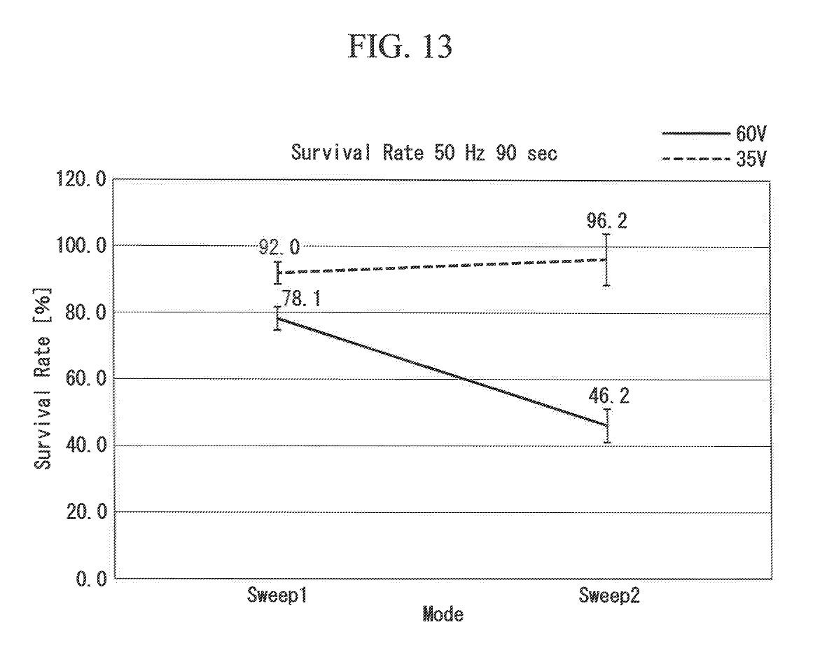

FIGS. 12 and 13 correspond to FIGS. 10 and 11, respectively, and illustrate the survival rate when the irradiation time is changed to 90 seconds. As seen from FIGS. 12 and 13, as with FIGS. 10 and 11, the survival rate at the ultrasonic wave output of 80 mW/cm² is lower than the survival rate at the ultrasonic wave output of 20 mW/cm², and there is no significant difference between Sweep 1 and Sweep 2 (the present disclosure) when the ultrasonic wave output is 35 V. On the other hand, as seen from FIG. 13, the survival rate can be decreased in Sweep 2 (the present disclosure), as long as the ultrasonic wave output is set to 80 mW/cm² and the pulse repetition frequency is set to 50 Hz, even when the irradiation time is 90 seconds.

In view of the above, the ultrasonic wave output should be equal to or more than 30 mW/cm², and preferably equal to or more than 80 mW/cm².

FIG. 14 illustrates the experimental results under the following conditions.

An ultrasonic wave was irradiated on cancer cells under the conditions that the center frequency was 510 kHz, the sweep width was ±110 kHz, the sweep interval was 0.2 ms, the sweep width was 22%, the pulse repetition frequency was 10 Hz, the duty ratio was 50%, the irradiation time was 180 seconds, and the ultrasonic wave output was 80 mW/cm². Furthermore, the cancer cells were placed in a static state for six hours in incubation in a humidified air environment with 5% CO₂ at 37° C. After the incubation period, the cancer cells were analyzed by TALI Image-Based Cytometer that measures the apoptosis and cell cycle.

As illustrated in FIG. 14, it was confirmed that the extent of early apoptosis and late apoptosis in Sweep 2 (the present disclosure) was more than that in Sweep 1, and the survival rate of cancer cells in Sweep 2 (the present disclosure) was lower than that in Sweep 1.

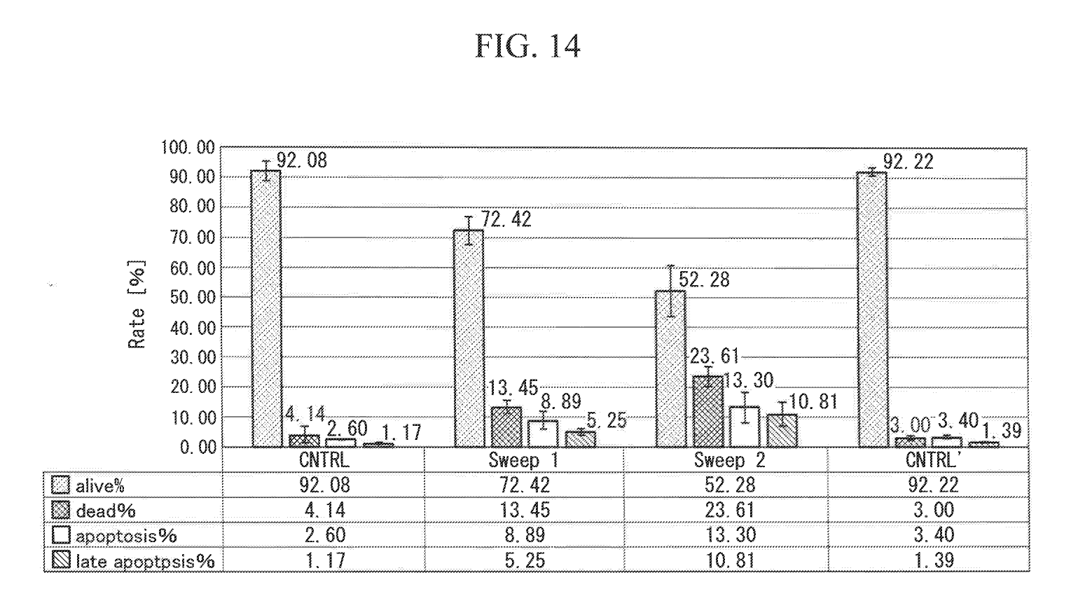

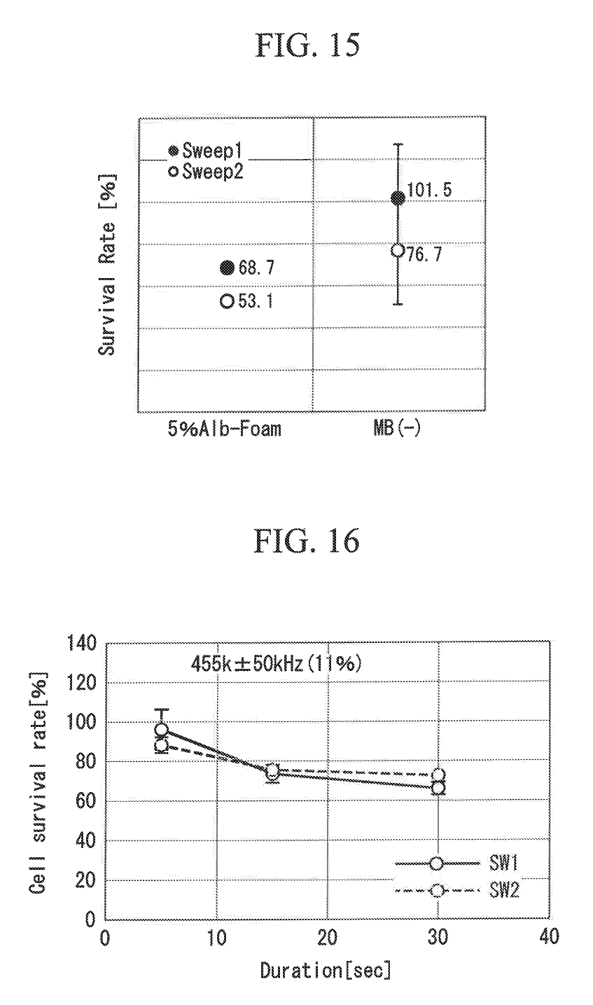

FIG. 15 illustrates the survival rate when microbubbles are used as an ultrasonic treatment accelerating substance. The left side in FIG. 15 illustrates the result of using microbubbles of albumin, and the right side of FIG. 15 illustrates the result of using no microbubbles. As seen from FIG. 15, the use of microbubbles allows the survival rate to be further decreased.

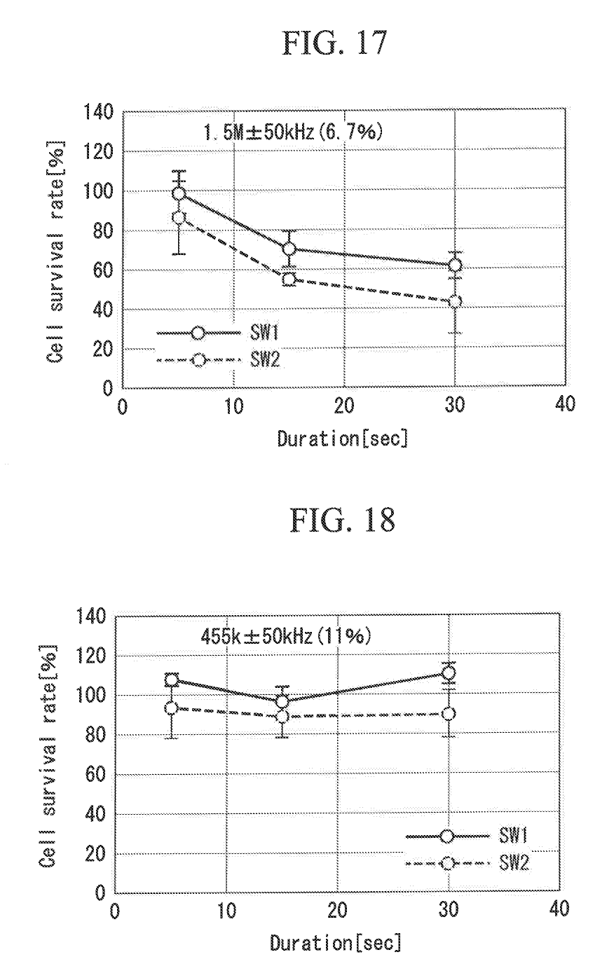

FIGS. 16 and 17 illustrate experimental results when Sonazoid MB (microbubble) is used.

A frequency sweep was carried out at a position where the frequency characteristic of the input impedance of the ultrasonic transducer is substantially flat. The experimental conditions are listed in the following table. An experimental condition (1) corresponds to FIG. 16, and an experimental condition (2) corresponds to FIG. 17.

TABLE 1

| | | | | |
|---|---|---|---|---|
| Experimental | Frequency | 455 kHz ± 50 kHz | Sweep | 1, 2 |
| Condition | Voltage | 60 V | Sweep width | 11% |
| (1) | Duty | 50% | Sweep interval | 0.2 ms |
| | Burst Rate | 50 Hz | Pulse type | Sine |
| | Duration | 5, 15, 30 sec | Bubble loads | 5 v/v % |
| Experimental | Frequency | 1.5 MHz ± 50 kHz | Sweep | 1, 2 |
| Condition | Voltage | 60 V | Sweep width | 6.7% |
| (2) | Duty | 50% | Sweep interval | 0.2 ms |
| | Burst Rate | 50 Hz | Pulse type | Sine |
| | Duration | 5, 15, 30 sec | Bubble loads | 5 v/v % |

As illustrated in FIG. 16, at the center frequency of 455 kHz, the cell killing effects of SW1 (which indicates Sweep 1, the same applying to the following) and SW2 (which indicates Sweep 2, the same applying to the following) corresponding to the present disclosure are substantially the same. However, as illustrated in FIG. 17, at the center frequency of 1.5 MHz, the cell killing effect in SW2 was better than that in SW1.

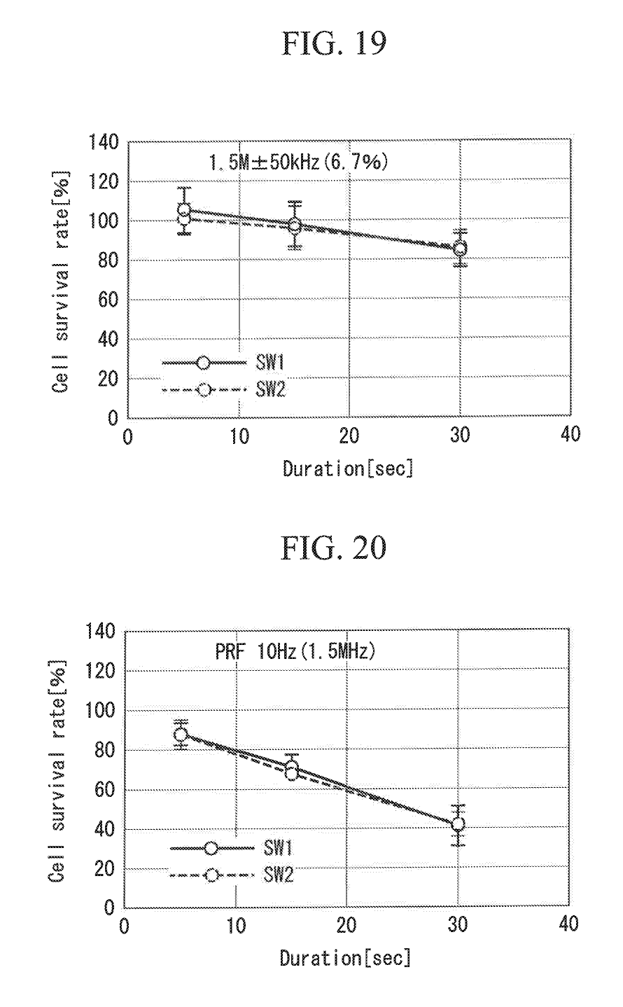

FIGS. 18 and 19 illustrate the experimental result when Sonazoid MB is not used unlike in FIGS. 16 and 17 and only the ultrasonic wave is used. The experimental conditions are listed below. An experimental condition (3) corresponds to FIG. 18, and an experimental condition (4) corresponds to FIG. 19.

TABLE 2

| | | | | |
|---|---|---|---|---|
| Experimental | Frequency | 455 kHz ± 50 kHz | Sweep | 1, 2 |
| Condition | Voltage | 60 V | Sweep width | 11% |
| (3) | Duty | 50% | Sweep interval | 0.2 ms |
| | Burst Rate | 100 Hz | Pulse type | Sine |
| | Duration | 5, 15, 30 sec | Bubble loads | None |
| Experimental | Frequency | 1.5 MHz ± 50 kHz | Sweep | 1, 2 |
| Condition | Voltage | 60 V | Sweep width | 6.7% |
| (4) | Duty | 50% | Sweep interval | 0.2 ms |
| | Burst Rate | 100 Hz | Pulse type | Sine |
| | Duration | 5, 15, 30 sec | Bubble loads | None |

As illustrated in FIG. 18, at the center frequency of 455 kHz, the cell killing effect in SW2 (the present disclosure) was better than that in SW1.

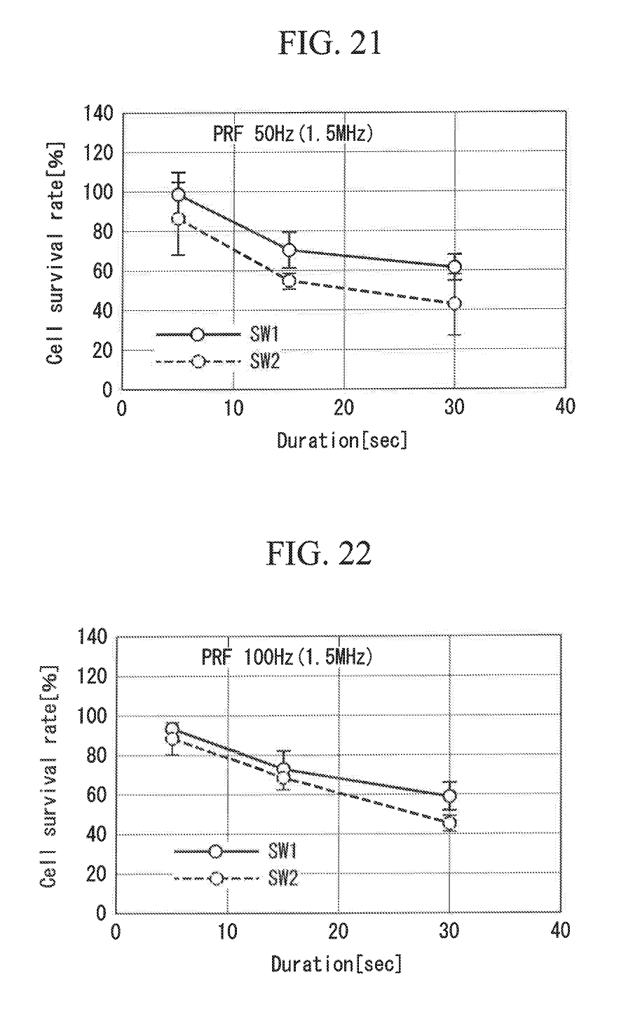

FIGS. 20 to 22 illustrate experimental results when Sonazoid MB is used and a PRF (pulse repetition frequency) is changed. The experimental results are listed in the following table.

TABLE 3

| | | | | |
|---|---|---|---|---|
| Experimental | Frequency | 1.5 MHz ± 50 kHz | Sweep | 1, 2 |
| Condition | Voltage | 60 V | Sweep width | 6.7% |
| (5) | Duty | 50% | Sweep interval | 0.2 ms |
| | Burst Rate | 10, 50, 100 Hz | Pulse type | Sine |
| | Duration | 5, 15, 30 sec | Bubble loads | 5 v/v % |

As illustrated in FIGS. 20 to 22, at 50 Hz in a range from PRF 10 to 100 Hz, the cell killing effect in SW2 (the present disclosure) was the best as compared with that in SW1.

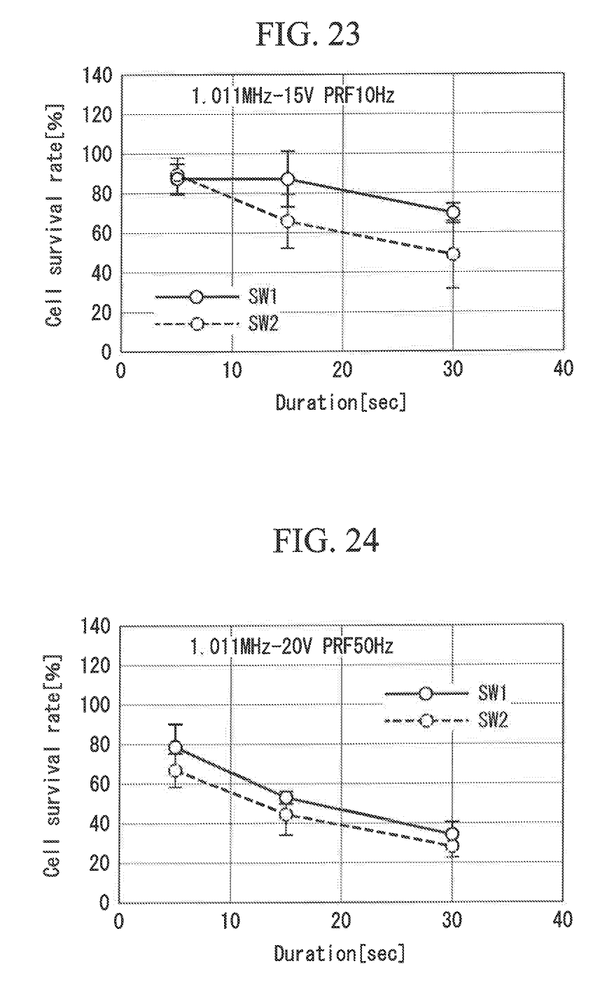

FIGS. 23 and 24 illustrate the experimental result when a frequency sweep was carried out using Sonazoid MB and with a peak of the frequency characteristic of the input impedance of the ultrasonic transducer as a center. The experimental results are listed in the following table.

TABLE 4

| | | | | |
|---|---|---|---|---|
| Experimental | Frequency | 1.011 MHz ± 50 kHz | Sweep | 1, 2 |
| Condition | Voltage | 15 V | Sweep width | 5% |
| (6) | Duty | 50% | Sweep | 0.2 ms |
| | | | interval | |
| | Burst Rate | 10 Hz | Pulse type | Sine |
| | Duration | 5, 15, 30 sec | Bubble loads | 5 v/v % |
| Experimental | Frequency | 1.011 MHz ± 50 kHz | Sweep | 1, 2 |
| Condition | Voltage | 20 V | Sweep width | 5% |
| (7) | Duty | 50% | Sweep | 0.2 ms |
| | | | interval | |
| | Burst Rate | 50 Hz | Pulse type | Sine |
| | Duration | 5, 15, 30 sec | Bubble loads | 5 v/v % |

As illustrated in FIGS. 23 and 24, the cell killing effect in SW2 (the present disclosure) was better than that in SW1. Note that in FIGS. 23 and 24, the input voltage was decreased to 15 V and 20 V, as compared with 60 V illustrated in FIGS. 16 to 22. This is because the input impedance of the ultrasonic transducer is excellent (small).

The following findings are obtained based on the experimental results illustrated in FIGS. 16 to 24.

The cell killing effect in SW2 (the present disclosure) was better than that in SW1.

At the center frequency of 1.5 MHz, the cell killing effect in SW2 at the input voltage of 60 V was excellent (see FIG. 17).

At the center frequency of 1.0111 MHz, the cell killing effect in SW2 at the input voltages of 15 V and 20 V was excellent (see FIGS. 23 and 24).

Furthermore, the uptake effect of dextran, which is an anticancer drug model, within cells was confirmed by flow cytometry. The experimental conditions are listed in the following table.

TABLE 5

| | | | | |
|---|---|---|---|---|
| Experimental | Frequency | 1.5 MHz ± 50 kHz | Sweep | 2 |
| Condition | Voltage | 60 V | Sweep width | 6.7% |
| (8) | Duty | 50% | Sweep interval | 0.2 ms |
| | Burst Rate | 50 Hz | Pulse type | Sine |
| | Duration | 15 sec | Bubble loads | 5 v/v % |
| | | | FITC-dextran | 50 μL/ |
| | | | 2000 kDa | well |
| | | | (6 mg/ml in | |
| | | | PBS) | |

As shown in the above table, when SW2 (the present disclosure) was carried out using Sonazoid MB, a possibility was suggested that dextran was taken up into cells due to the FITC fluorescence intensity.

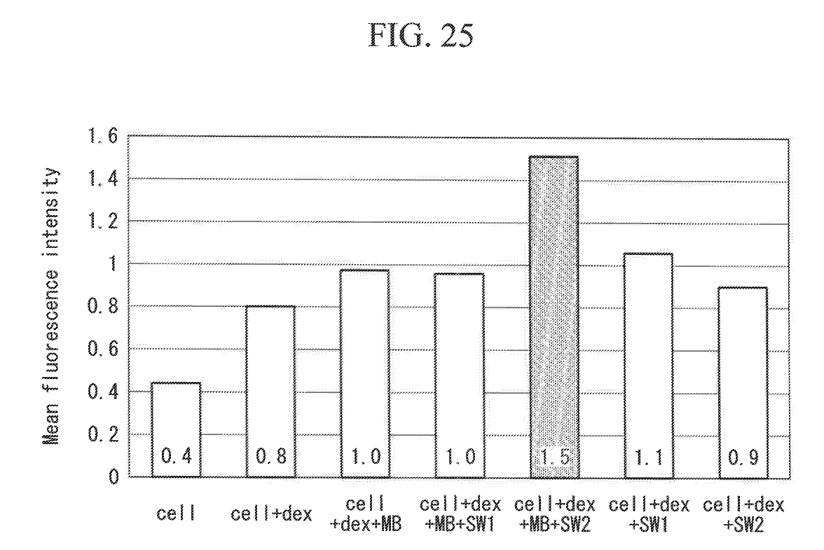

Furthermore, as illustrated in FIG. 25, when the average value of the FITC fluorescence intensities was obtained under the experimental conditions in the following table, it was recognized that the average value when SW2 (the present disclosure) was carried out using Sonazoid MB was highest. This suggested a possibility that dextran was taken up into cells by using SW2.

TABLE 6

| | | | | |
|---|---|---|---|---|
| Experimental | Frequency | 1.5 MHz ± 50 kHz | Sweep | 1, 2 |
| Condition | Voltage | 60 V | Sweep width | 6.7% |
| (9) | Duty | 50% | Sweep interval | 0.2 ms |
| | Burst Rate | 50 Hz | Pulse type | Sine |
| | Duration | 30 sec | Bubble loads | 5 v/v % |
| | | | FITC-dextran | 5 μL/ |
| | | | 2000 kDa | well |
| | | | (6 mg/ml in | |
| | | | PBS) | |

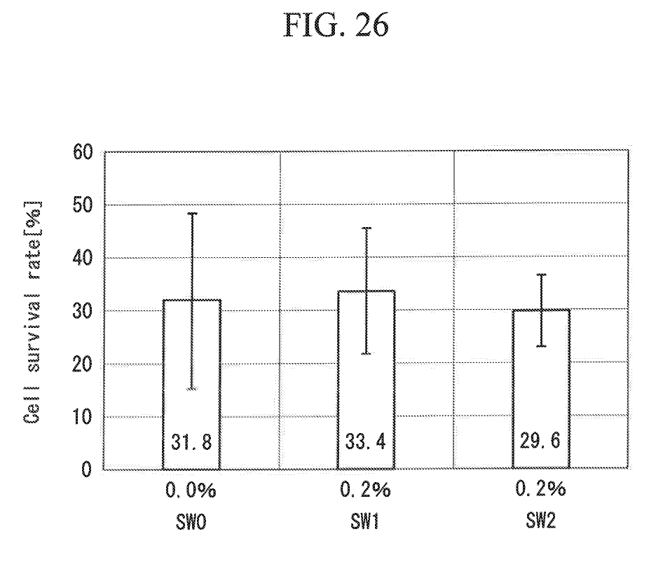

FIG. 26 illustrates experimental results when the presence or absence of a frequency sweep is changed using a peak (i.e., a resonance frequency of 1.011 MHz) of the frequency characteristic of the input impedance of each ultrasonic transducer. Experimental results are shown in the following table. SW0 indicates that there is no frequency sweep.

TABLE 7

| | | | | |
|---|---|---|---|---|
| Experimental | Frequency | 1.011 MHz | Sweep | 0, 1, 2 |
| Condition | Voltage | 20 V | Sweep width | 0, 0.2% |
| (10) | Duty | 50% | Sweep interval | 0.2 ms |
| | Burst Rate | 50 Hz | Pulse type | Sine |
| | Duration | 15 sec | Sonazoid loads | 5 v/v % |

As illustrated in FIG. 26, when the case where no frequency sweep was carried out (SW0) is compared with the case where a frequency sweep was carried out (SW1, SW2), the cell killing effect was substantially the same. However, a standard deviation (corresponding to an error bar in FIG. 26) representing a variation in data between a plurality of ultrasonic transducers in the case where a frequency sweep was carried out (SW1, SW2) was smaller than that in the case where no frequency sweep was carried out (SW0), and the standard deviation in SW2 (the present disclosure) was smaller than that in SW1. As a result, when ultrasonic transducers are driven with a resonance frequency, a variation in data between the plurality of ultrasonic transducers can be suppressed by using the frequency sweep.

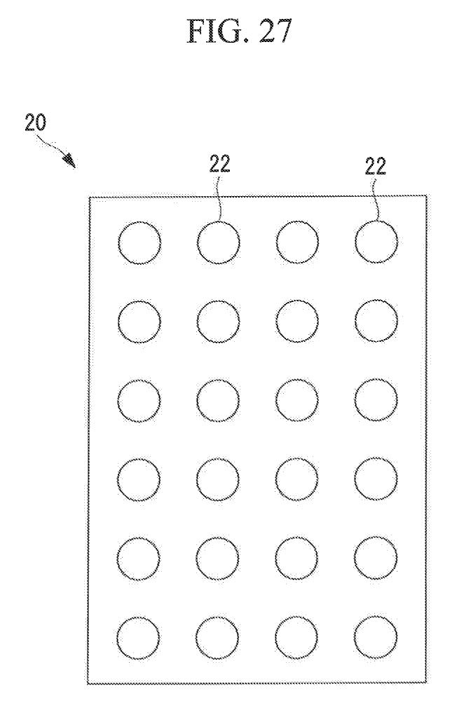

FIG. 27 illustrates a pallet 20 used when the experiments described above are conducted. The pallet 20 is provided with a plurality of wells 22, and the wells 22 are placed in, for example, six rows and four columns.

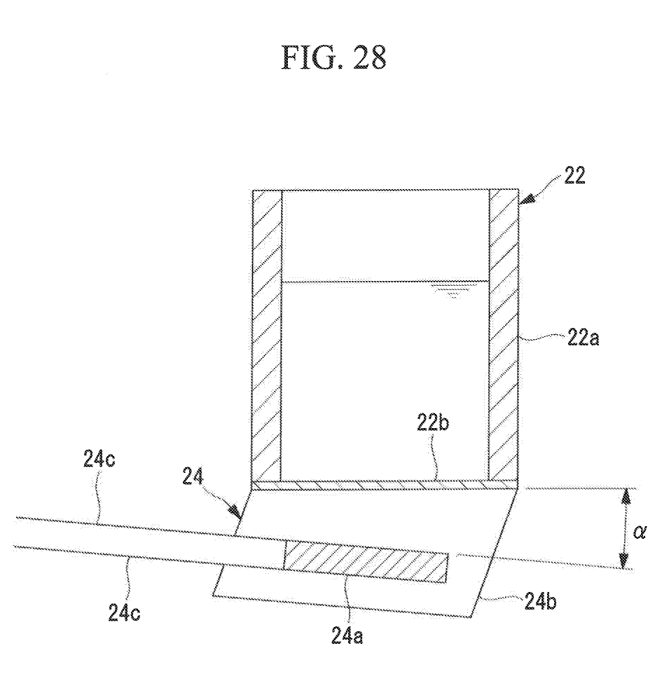

The well 22 is a cylindrical container as illustrated in FIG. 28 as an enlarged view of one well 22. The well 22 includes a cylindrical side wall part 22a, a film 22b is liquid tightly fixed to a bottom part of the side wall part 22a. The film 22b is a resin thin film that is likely to transmit ultrasonic waves. The inside of the well 22 is filled with an aqueous solution to which Sonazoid MB or dextrin is added.

Ultrasonic transducers 24 are disposed on the film 22b, which constitutes the bottom part of the well 22, in such a manner that the ultrasonic transducers are in contact with the film during experiments. The ultrasonic transducer 24 includes a vibration element 24a, an accommodation body 24b that is provided so as to surround the vibration element 24a and is filled with water (liquid), and an electric wire 24c that supplies electric power to the vibration element 24a. The ultrasonic transducers 24 are provided so as to irradiate an ultrasonic wave from a direction inclined with respect to the surface of the film 22b (direction inclined by the angle α with respect to the surface of the film 22b). This configuration prevents the irradiated ultrasonic wave from interfering with the ultrasonic wave reflected on the surface of the film 22b and forming a standing wave, and improves the energy permeability with respect to the film 22b.

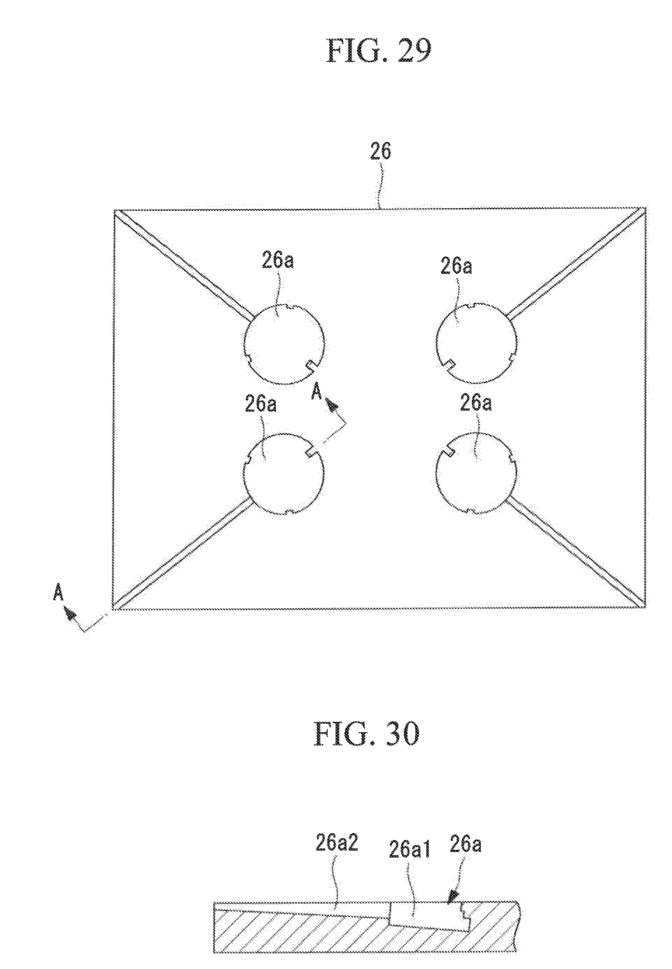

FIG. 29 illustrates an experimental jig 26 that accommodates the ultrasonic transducers 24. The experimental jig 26 has a plate-like body, and four grooves 26a are formed in the surface of the experimental jig so that four ultrasonic transducers 24 can be installed.

As illustrated in FIG. 30, each groove 26a includes a circular groove 26a1 that accommodates the accommodation body 24b (see FIG. 28) of the corresponding ultrasonic transducer 24, and a lead groove 26a2 that communicates with the circular groove 26a1 and linearly extends. The electric wire 24c (see FIG. 28) is accommodated in the lead groove 26a2. The pallet 20 (see FIG. 27) is installed on the surface of the experimental jig 26, thereby allowing the ultrasonic transducers 24 to be disposed obliquely with respect to the film 22b of the bottom surface of the well 22 as illustrated in FIG. 28.

As described above, according to this embodiment, the following operation and effect are obtained.

The plurality of ultrasonic transducers 10 are disposed on the placement surface, which is the inner surface 7a of the sphere, so that the focal point F is located at a position that is near the outside of the outer surface S of the subject M and is spaced apart from the outer surface by the predetermined distance A. With this configuration, the ultrasonic wave emitted from each of the ultrasonic transducers 10 can be prevented from being concentrated on one location on the outer surface of the subject M or within the body of the subject M, so that there is no thermal adverse effect, such as burn, on the subject.

Furthermore, the focal point F is formed at a predetermined position near the outside of the outer surface of the subject M, thereby reducing the ultrasonic wave irradiation region on the outer surface S of the subject M as much as possible, and making it possible to perform diagnosis or treatment by using all the ultrasonic transducers 10 effectively even when the region through which the ultrasonic wave can be irradiated on the subject is limited.

Since the spread angle α of the ultrasonic wave at the focal point F is set in a range from 80° to 160°, the ultrasonic wave can be irradiated with the sufficient spread angle α toward the inside of the body of the subject M and a wide diagnosis region and a wide treatment region of the subject M can be set. In particular, the use of the ultrasonic transducer 10k with the large incident angle β with respect to the outer surface S of the subject M (see FIG. 6) enables the ultrasonic wave to reach the treatment target T that is located on the outside of the irradiation region on the outer surface S and at a shallow depth from the outer surface.

Note that the above embodiments can be modified as follows.

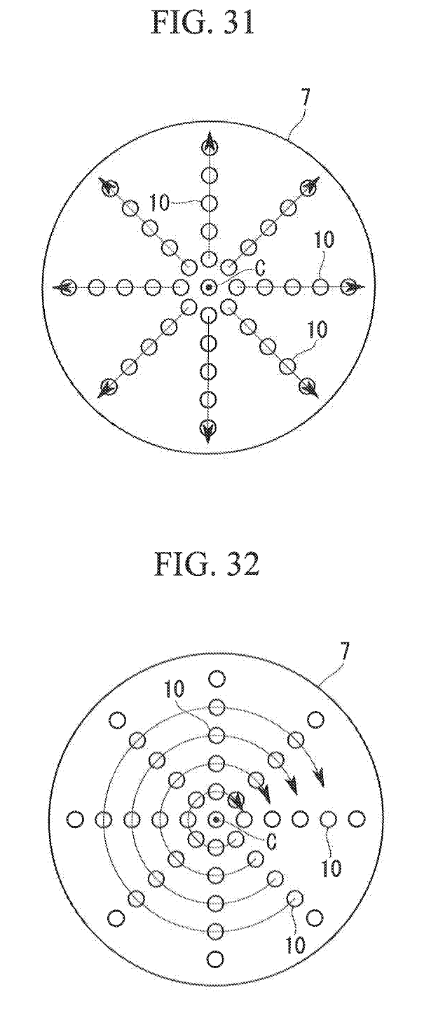

The sequence of oscillation of the ultrasonic transducers 10 is not limited to the diameter direction of the ultrasonic wave transmission/reception part 7 described above with reference to FIG. 3. The ultrasonic transducers 10 may be sequentially oscillated in the radial direction from the center C as illustrated in FIG. 31, or may be sequentially oscillated in the circumferential direction as illustrated in FIG. 32.

Not only in the treatment mode, but also in the diagnosis mode, all ultrasonic transducers 10 or some of the ultrasonic transducers 10 may be simultaneously oscillated as long as the reflected wave can be separated (e.g., the oscillation frequency of each ultrasonic transducer 10 is varied).

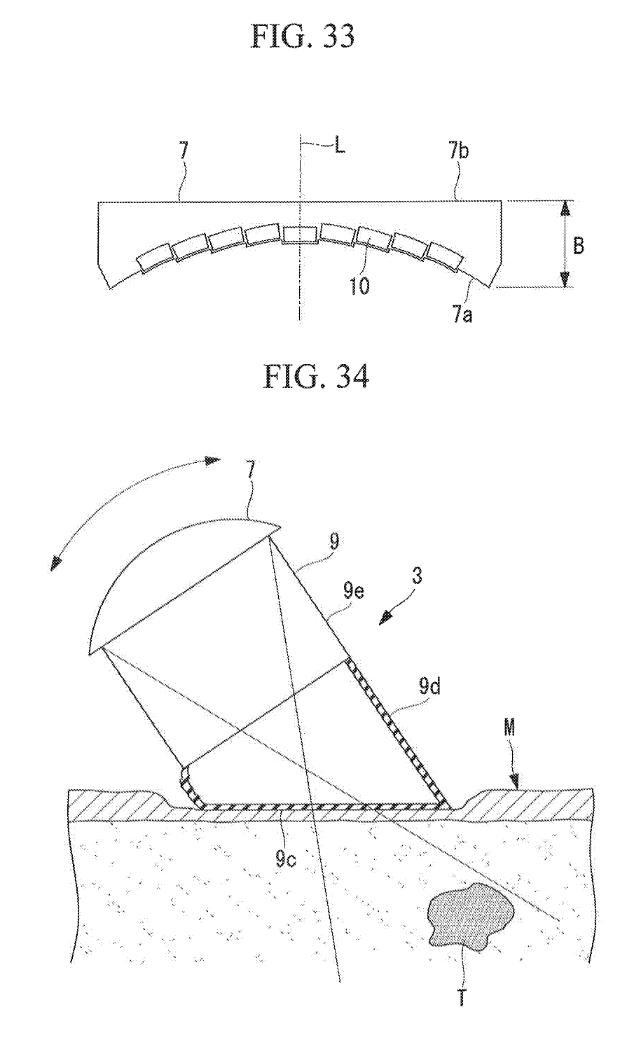

As illustrated in FIG. 33, the installation angle of each ultrasonic transducer 10 may be inclined more than the inclination angle of the inner surface 7a. More specifically, the ultrasonic transducers 10 are installed in such a manner that the ultrasonic transducers face the central axis L as spaced apart from the central axis L. Thus, by reducing the curvature radius of the inner surface 7a, a thickness B of the ultrasonic wave transmission/reception part 7 that supports the ultrasonic transducers 10 can be reduced and the device can be made compact.

Furthermore, as illustrated in FIG. 34, a deformable contact part 9d that is formed of an elastic material, such as rubber, which is elastically deformable, may be provided at a lower part of the coupling part 9. The deformable contact part 9d has an outside diameter similar to that of a main body part 9e of the upper coupling part 9. In a state where no external force is applied, the deformable contact part 9d has a bottomed cylindrical cup shape having a side surface with a height (e.g., about 3 cm) in the axial direction that coincides with the circumferential direction. The inside of the deformable contact part 9d is filled with an acoustic matching liquid such as deaerated water or silicone oil. In this case, the inside of the deformable contact part 9d may communicate with the main body part 9e of the upper coupling part 9, and a common acoustic matching liquid may be used.

As illustrated in FIG. 34, when the probe 3 is inclined with respect to the subject M, the bottom part 9c of the deformable contact part 9d can be deformed while being in contact with the subject M according to the inclination, and the ultrasonic wave can reach the treatment target T that is located immediately below a bone, so that diagnosis and treatment can be performed satisfactorily.

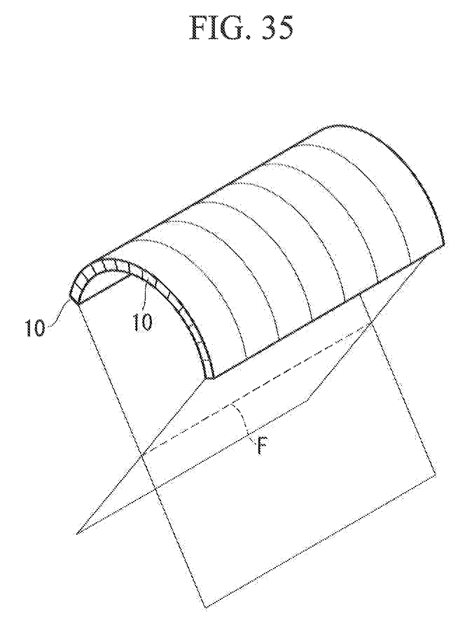

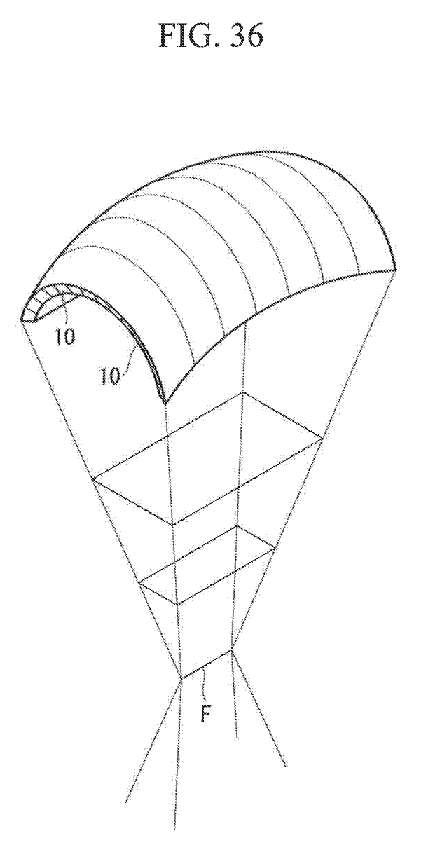

FIGS. 35 and 36 illustrate modified examples of the placement of the ultrasonic transducers 10. In the embodiments described above, the ultrasonic transducers 10 are placed on the inner surface 7a (e.g., see FIG. 2) having a spherical shape, but the present disclosure is not limited to this. For example, as illustrated in FIG. 35, the plurality of ultrasonic transducers 10 may be placed in a cylindrical surface shape. Furthermore, as illustrated in FIG. 36, the plurality of ultrasonic transducers 10 may be placed in an elliptic surface shape. Note that reference symbol F illustrated in FIGS. 35 and 36 denotes a focal point.

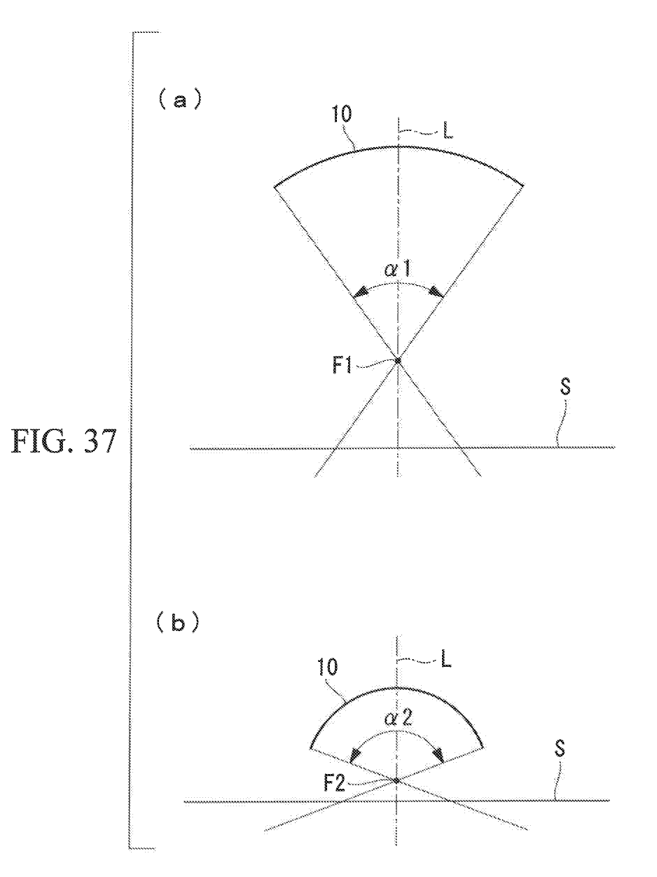

The inner surface 7a may be provided with an ultrasonic element placement shape change part that deforms the ultrasonic wave transmission/reception part 7, which holds the plurality of ultrasonic transducers 10, to change the placement shape of the ultrasonic transducers 10. Specifically, as illustrated in FIG. 37(a), deformation can be made from a state where the curvature radius of the inner surface on which the ultrasonic transducers 10 are placed is increased, to a state where the curvature radius of the inner surface on which the ultrasonic transducers 10 are placed as illustrated in FIG. 37(b) is reduced. The length of the arc of the inner surface on which the ultrasonic transducers 10 are placed does not change greatly. Accordingly, when deformation is made so as to reduce the curvature radius as illustrated in FIGS. 37(a) to 37(b), the spread angle can be increased from α1 to α2. Furthermore, the focal point position is closer to the outer surface S from F1 to F2. Accordingly, when the ultrasonic transducers 10 are made close to the outer surface S as illustrated in FIGS. 37(a) to 37(b), the ultrasonic wave can be transmitted and received with a large spread angle.

Examples of the mechanism of the ultrasonic element placement shape change part that changes the curvature radius of the inner surface on which the ultrasonic transducers 10 are placed are given below.

The ultrasonic wave transmission/reception part 7 is formed of an elastic material, such as resin or rubber, which is elastically deformable at an ordinary temperature, and the plurality of ultrasonic transducers 10 is placed on the inner surface 7a of the elastically deformable ultrasonic wave transmission/reception part 7 (see FIG. 2). A frame, such as an umbrella, is attached to the ultrasonic wave transmission/reception part 7. Specifically, the frame is formed of a plurality of elastic members radially extending along the outer surface from the vertex of the ultrasonic wave transmission/reception part 7 having a substantially hemispherical shape. Each elastic member is a rod-like member that is obtained by, for example, stacking a plurality of piezoelectric elements in the longitudinal direction, and is bent at a predetermined curvature radius. Each piezoelectric element is energized to be stretched and thus the elastic member is also stretched, and the curvature radius is changed. The piezoelectric elements are stacked so that the curvature radius of the elastic member is increased when the elastic member is stretched, thereby enabling deformation from a state where energization is not performed and the curvature radius is small (see FIG. 37(b)), to a state where energization is performed and the curvature radius is large (see FIG. 37(a)). The state of energization to the piezoelectric elements is controlled by the control part 5 (see FIG. 1). Note that the mechanism for changing the curvature radius of the inner surface on which the ultrasonic transducers 10 are placed is not limited to the frame structure using the piezoelectric elements described above. For example, an actuator using an oil pressure or a hydraulic pressure, or other mechanisms may be used.

Thus, the curvature radius of the inner surface on which the ultrasonic transducers 10 are placed is arbitrarily changed according to a command from the control part 5 depending on an area on which diagnosis or treatment is performed, and the spread angles α1 an α2 are adjusted, thereby ensuring a desired field-of-view range or treatment range.

### REFERENCE SIGNS LIST

1 ultrasonic diagnosis/treatment device
2 ultrasonic diagnosis/treatment probe
5 control part
7 ultrasonic wave transmission/reception part
7a inner surface
7b packing part
9 coupling part
9a in-flow port
9b out-flow port
9c bottom surface
9d deformable contact part
9e main body part
10 ultrasonic transducer
F focal point
L central axis
M subject
S Outer surface
T treatment target

1. An ultrasonic diagnosis/treatment device comprising:
a plurality of ultrasonic transducers placed in such a manner that a focal point of an ultrasonic wave to be emitted is located at a position that is near an outside of an outer surface of a subject and is spaced apart from the outer surface by a predetermined distance; and
a control part configured to control transmission and reception of each of the ultrasonic transducers, wherein
the control part includes:
a diagnosis mode in which at least one of the ultrasonic transducers is oscillated toward a diagnosis region within the subject and a reflected wave from the diagnosis region is received by the at least one of the ultrasonic transducers to visualize the reflected wave; and
a treatment mode in which the at least one of the ultrasonic transducers is oscillated toward an inside of the subject.

2. The ultrasonic diagnosis/treatment device according to claim 1, wherein the plurality of ultrasonic transducers is placed on a placement surface, the placement surface being a concave curve.

3. The ultrasonic diagnosis/treatment device according to claim 1, wherein the predetermined distance is in a range from 3 mm to 30 mm.

4. The ultrasonic diagnosis/treatment device according to claim 1, wherein in the diagnosis mode, the control part causes each of the ultrasonic transducers to be sequentially oscillated at a different time or at a different phase.

5. The ultrasonic diagnosis/treatment device according to claim 1, wherein a maximum spread angle of the ultrasonic wave at the focal point is in a range from 80° to 160°.

6. The ultrasonic diagnosis treatment device according to claim 1, wherein in the diagnosis mode, the control pert selects at least one of the ultrasonic transducers to irradiate an ultrasonic wave in a direction opposite to a movement direction of a flow of blood flowing within a blood vessel, oscillates the selected ultrasonic transducer toward the blood vessel to obtain ultrasonic wave Doppler, and measures a rate of the flow of the blood.

7. The ultrasonic diagnosis/treatment device according to claim 1, wherein in the treatment mode, the control part causes at least one of the ultrasonic transducers to be oscillated toward a treatment position where a drug and an ultrasonic treatment accelerating substance are administered, the at least one of the ultrasonic transducers corresponding to the treatment position.

8. The ultrasonic diagnosis/treatment device according to claim 1, further comprising a deformable contact part that is elastically deformable while being in contact with a surface of the subject.

9. The ultrasonic diagnosis/treatment device according to claim 1, wherein in the treatment mode, the control part includes a frequency sweep mode in which a frequency included in a pulse wave oscillated from the ultrasonic transducers is swept from a high frequency region to a low frequency region.

10. The ultrasonic diagnosis/treatment device according to claim 1, further comprising an ultrasonic transducer placement shape change part configured to change a placement shape of the ultrasonic transducers so as to change a maximum spread angle of the ultrasonic wave at the focal point.

11. A ultrasonic diagnosis/treatment method using an ultrasonic diagnosis/treatment device including a plurality of ultrasonic transducers placed in such a manner that a focal point of an ultrasonic wave to be emitted is located at a position that is near an outside of an outer surface of a subject and is spaced apart from the outer surface by a predetermined distance, the ultrasonic diagnosis/treatment method comprising:
performing a diagnosis mode in which at least one of the ultrasonic transducers is oscillated toward a diagnosis region within the subject and a reflected wave from the diagnosis region is received by the at least one of the ultrasonic transducers to visualize the reflected wave; and
performing a treatment mode in which the at least one of the ultrasonic transducers is oscillated toward an inside of the subject.
